(19) United States
(12) Patent Application Publication
(10) Pub. No.: US 2015/0148524 A1
(43) Pub. Date: May 28, 2015

(54) AMINO ACID ANALOGUES AND METHODS FOR THEIR SYNTHESIS

(71) Applicant: MONASH UNIVERSITY, Clayton (AU)

(72) Inventors: Zhen Wang, Victoria (AU); Andrea Robinson, St. Kilda (AU); Nicolas Daniel Spiccia, Victoria (AU); William Roy Jackson, Camberwell (AU)

(21) Appl. No.: 14/409,509

(22) PCT Filed: Jul. 8, 2013
(86) PCT No.: PCT/AU2013/000747

§ 371 Date: Dec. 19, 2014

(30) Foreign Application Priority Data
Jul. 6, 2012 (AU) 2012902916

(51) Int. Cl. C07K 1/00; C07D 255/02; C07K 2/00; C07C 269/06

(52) U.S. Cl. CPC C07K 1/006; C07C 269/06; C07D 255/02; C07K 2/00
USPC 530/337; 560/163; 560/157; 552/544; 540/474; 560/160; 554/110; 530/300

(57) ABSTRACT

A method for the synthesis of an amino acid analogue or a salt, solvate, derivative, isomer or tautomer thereof comprising the steps of: (i) subjecting an amino acid containing a metathesisable group to metathesis with a compound containing a complementary metathesisable group of formula (I) or (II): (Formulae (I), (II)) wherein R¹ and R² are independently selected from H and substituted or unsubstituted C₁ to C₄ alkyl; each R³ is either absent or independently selected from a heteroatom, a substituted or unsubstituted C₁ to C₂₀ alkyl, and a substituted or unsubstituted C₁ to C₂₀ alkyl group interrupted by one or more heteroatoms; and each X is independently selected from H and an effector molecule; in the presence of a reagent to catalyse the metathesis to form a dicarba bridge between the amino acid containing a metathesisable group and the compound containing a complementary metathesisable group; and (ii) reducing the dicarba bridge to form a saturated dicarba bridge, wherein the reagent used to catalyse step (i) also catalyses step (ii).

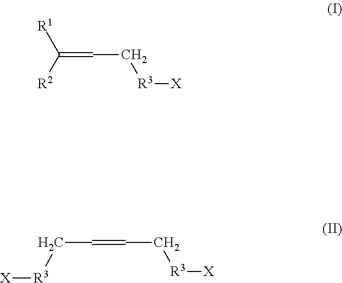

### FIELD

The present invention broadly relates to methods for the synthesis of amino acid analogues, and peptides containing them. The present invention also relates to amino acid analogues per se and peptides containing them.

### BACKGROUND

α-Amino acids analogues having extended alkyl sidechains are known as lipidic amino acids (LAAs). Preparation of these amino acid analogues has previously been performed by racemic syntheses, which involve the combination of two reactants at the α-carbon. The reactants which can be used in these types of reactions are limited, in some instances to shorter side chains due to poor substrate tolerance. The racemic mixture produced by these methods must then be separated by subsequent steps of diastereomeric resolution to isolate the chiral amino acid analogues Enzymatic or chemical resolution methods are not trivial and often result in significant losses in yield.

While asymmetric synthetic methods have been tried, the yields have been poor and have required the use of expensive chiral auxiliaries to access the L- or D-configured amino acid analogues. At the end of the process, undesirable chromatographic purification is also required to arrive at a product suitable for use in solid phase peptide synthesis.

Current research involving amino acid analogues and peptidomimetics containing LAAs is hampered by the high commercial cost and limited availability of the amino acid analogues. There is therefore a need for a method of preparing LAAs and peptides containing them in an efficient manner, and without the need for resolution.

### SUMMARY

The present invention relates to a method for the synthesis of chiral amino acid analogues without the need for resolution. The method involves an olefin metathesis step, in which the stereogenic centre (the α-carbon) adjacent to the carboxyl functionality is not epimerised during the metathesis reaction, thereby eliminating the need for diastereomeric resolution. The unsaturated amino acid analogues may be subsequently reduced to prepare saturated amino acid analogues. Preferably, the catalyst from the metathesis step is used to catalyse the subsequent reduction step.

The method of the present invention preferably utilises commercially available starting materials and requires minimal protecting group manipulation. Furthermore, the amino acid analogues can be produced by the method in a one or two step synthesis, and may be incorporated into a peptide sequence without further purification. Greater accessibility to these amino acid analogues also simplifies the preparation of modified peptides and peptidomimetics.

In a first aspect, there is provided a method for the synthesis of an amino acid analogue or a salt, solvate, derivative, isomer or tautomer thereof comprising the steps of:

(i) subjecting an amino acid containing a metathesisable group to metathesis with a compound containing a complementary metathesisable group of formula (I) or (II):

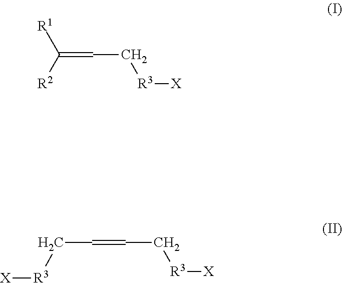

wherein
R¹ and R² are independently selected from H and substituted or unsubstituted C₁ to C₄ alkyl;
each R³ is either absent or independently selected from a heteroatom, a substituted or unsubstituted C₁ to C₂₀ alkyl, and a substituted or unsubstituted C₁ to C₂₀ alkyl group interrupted by one or more heteroatoms; and
each X is independently selected from H and an effector molecule;
in the presence of a reagent to catalyse the metathesis to form a dicarba bridge between the amino acid containing a metathesisable group and the compound containing a complementary metathesisable group; and
(ii) reducing the dicarba bridge to form a saturated dicarba bridge.

In a second aspect, there is provided an amino acid analogue or a salt, solvate, derivative, isomer or tautomer thereof synthesised by the method as described above.

In a third aspect, there is provided a method for preparing a peptide containing an amino acid residue of formula (X¹) or a salt, solvate, derivative, isomer or tautomer thereof,

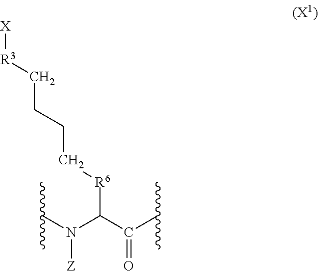

comprising the steps of:
(i) subjecting an amino acid containing a metathesisable group of formula (VI) to metathesis with a compound containing a complementary metathesisable group of formula (I) or (II):

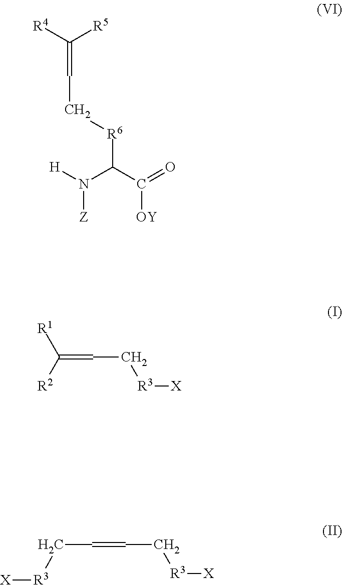

wherein R¹, R², R⁴ and R⁵ are independently selected from H and substituted or unsubstituted C₁ to C₄ alkyl; each R³ is either absent or independently selected from a heteroatom, a substituted or unsubstituted C₁ to C₂₀ alkyl, and a substituted or unsubstituted C₁ to C₂₀ alkyl group interrupted by one or more heteroatoms; R⁶ is either absent or selected from a heteroatom, a substituted or unsubstituted C₁ to C₂₀ alkyl, and a substituted or unsubstituted C₁ to C₂₀ alkyl interrupted by one or more heteroatoms; Z is selected from H, a salt and a protecting group; Y is selected from H and a protecting group; and each X is independently selected from H and an effector molecule; in the presence of a reagent to catalyse the metathesis to form a dicarba bridge between the amino acid of formula (VI) and the compound of formula (I) or (II);
(ii) reducing the dicarba bridge to form a saturated dicarba bridge; and
(iii) synthesising a peptide by stepwise addition of amino acid residues to produce the peptide, wherein one or more of the amino acid residues is of formula (X¹).

In a fourth aspect, there is provided a peptide or a salt, solvate, derivative, isomer or tautomer thereof synthesised by the method as described above.

In a fifth aspect, there is provided a method for the synthesis of a peptide or peptides containing a dicarba bridge or a salt, solvate, derivative, isomer or tautomer thereof comprising the steps of:

(i) subjecting a reactable peptide containing a metathesisable group to metathesis with a compound containing a complementary metathesisable group of formula (I′) or (II′):

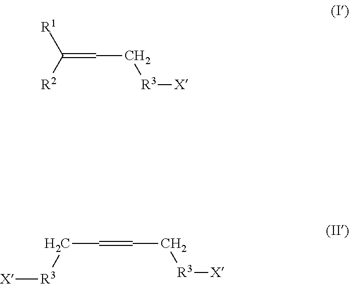

wherein
R¹ and R² are independently selected from H and substituted or unsubstituted C₁ to C₄ alkyl;
each R³ is either absent or independently selected from a heteroatom, a substituted or unsubstituted C₁ to C₂₀ alkyl, and a substituted or unsubstituted C₁ to C₂₀ alkyl group interrupted by one or more heteroatoms; and
each X′ is independently selected from H, an effector molecule, an amino acid and a peptide;
in the presence of a reagent to catalyse the metathesis to form a dicarba bridge between the reactable peptide containing a metathesisable group and the compound containing a complementary metathesisable group; and
(ii) reducing the dicarba bridge to form a saturated dicarba bridge.

In a preferred embodiment of the first, third and fifth aspect, the reagent used to catalyse step (i) also catalyses step (ii), the reduction step.

The present inventors have surprisingly found that the reagent which has been used to catalyse the metathesis reaction can perform the required reduction reaction under mild conditions. Experimental conditions used for reduction following olefin metathesis typically employ harsh conditions. For example, reduction is typically carried out under high pressure (up to 30 bar, which is equivalent to about 440 Psi), at high temperatures (such as reflux and 150° C.) and/or in the presence of a base (such as sodium hydride, lithium aluminium hydride, calcium hydride, sodium hydroxide, potassium carbonate, sodium hydroxide, potassium hydroxide, potash, potassium tert-butoxide or ammonia). The present inventors have found that the catalyst residue following metathesis can perform the required reduction reaction under mild hydrogen pressure, low temperature and without the addition of a base or an additional reagent to catalyse the reduction.

The use of a base, particularly a strong base, is unsuitable for amino acid and peptide substrates. Therefore, identification of tandem metathesis-reduction via a single catalyst under mild experimental conditions, such as low pressure and temperature and in the absence of a base, provides a practical method for the synthesis of chiral lipophilic amino acids and peptides containing them.

The reagent used to catalyse the metathesis and the reduction may be referred to herein as a “recycled metathesis catalyst”, and the process involving a metathesis step immediately followed by a reduction step via a single catalyst is herein referred to as “tandem metathesis-reduction” or “tandem metathesis-hydrogenation”, which phrases may be used interchangeably. Using the tandem metathesis-hydrogenation method there is no need for work-up of the metathesis product prior to reduction step, which simplifies the method and avoids losses in the target product which may result from separate work-up steps after metathesis and after reduction. The recycled metathesis catalyst can also be left in air for several days and still generate an active reduction catalyst.

In a sixth aspect, there is provided a method for the synthesis of a peptide containing a dicarba bridge or a salt, solvate, derivative, isomer or tautomer thereof comprising the steps of:

(i) subjecting a reactable peptide containing at least two metathesisable groups to metathesis in the presence of a reagent to catalyse the metathesis to form a dicarba bridge between the metathesisable groups of the reactable peptide; and
(ii) reducing the dicarba bridge to form a saturated dicarba bridge,
wherein the reagent used to catalyse step (i) also catalyses step (ii).

In a seventh aspect, there is provided a peptide or peptides or a salt, solvate, derivative, isomer or tautomer thereof synthesised by the method as described above.

### DETAILED DESCRIPTION

As described above, the present invention relates to a method for the synthesis of amino acid analogues or peptides containing the amino acid analogues or salts, solvates, derivatives, isomers or tautomers thereof, by tandem metathesis-reduction. In the method, the stereogenic centre adjacent to the carbonyl functionality (the α-carbon) is not epimerised during the metathesis reaction, thereby eliminating the need for diastereomeric resolution. The unsaturated dicarba bridge-containing amino acid analogues or peptides containing them are subsequently reduced to prepare saturated dicarba bridge-containing amino acid analogues or peptides containing them using the same catalyst for the metathesis and subsequent reduction. The amino acid analogues can be used directly in peptide synthesis to produce peptides containing the amino acid analogues.

The method utilises commercially available starting materials and requires minimal protecting group manipulation. Therefore, the amino acid analogues or peptides containing them can be produced without the need for work-up of the metathesis product prior to the reduction step. Furthermore, using the tandem metathesis-reduction method the required reduction reaction can be conducted under mild conditions which are compatible with amino acid and peptide substrates. Specifically, the catalyst residue following metathesis can perform the required reduction reaction under mild hydrogen pressure (for example, 100 Psi or less), low temperature (for example, below 100° C., and preferably at room temperature) and without the addition of a base or an additional reagent to catalyse the reduction. The use of a base, particularly a strong base is unsuitable for amino acid and peptide substrates, and therefore the present method involving tandem metathesis-reduction using a single catalyst under mild experimental conditions is essential for accessing chiral lipidic amino acids such as the amino acids of the present invention, and peptides containing them.

This method provides an improved route for preparation of amino acid analogues, and for the preparation of modified peptides and peptidomimetics containing them.

### DEFINITIONS

The term “amino acid” is used in its broadest sense and refers to L- and D-amino acids including the 20 common amino acids such as alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine; and the less common amino acid derivatives such as homo-amino acids (e.g. β-amino acids), N-alkyl amino acids, dehydroamino acids, aromatic amino acids and α,α-disubstituted amino acids, for example, cystine, 5-hydroxylysine, 4-hydroxyproline, α-aminoadipic acid, α-amino-n-butyric acid, 3,4-dihydroxyphenylalanine, homoserine, α-methylserine, ornithine, pipecolic acid, ortho, meta or para-aminobenzoic acid, citrulline, canavanine, norleucine, δ-glutamic acid, aminobutyric acid, L-fluorenylalanine, L-3-benzothienylalanine and thyroxine; β-amino acids (as compared with the typical α-amino acids) and any amino acid having a molecular weight less than about 500. The term amino acids can also include non-natural amino acids such as those described in U.S. Pat. No. 6,559,126, which is incorporated herein by reference. The term also encompasses amino acids in which the side chain of the amino acid comprises a metathesisable group, as described herein. Further, the amino acid may be a pseudoproline residue (ψPro).

The term “side chain” is used in the usual sense to refer to the side chain on the amino acid, and the backbone to the H₂N—(C)ₓ—CO₂H (where x=1, 2 or 3) component, in which the carbon in bold text bears the side chain (the side chain being possibly linked to the amino nitrogen, as in the case of proline).

The term “optionally protected” is used herein in its broadest sense and refers to an introduced functionality which renders a particular functional group, such as a hydroxy, amino, carbonyl or carboxyl group, unreactive under selected conditions and which may later be optionally removed to unmask the functional group. A protected amino acid is one in which the reactive substituents of the amino acid, the amino group, carboxyl group or side chain of the amino acid are protected. Suitable protecting groups are known in the art and include those disclosed in Greene, T. W., “Protective Groups in Organic Synthesis” John Wiley & Sons, New York 1999 (the contents of which are incorporated herein by reference) as are methods for their installation and removal.

By a “peptide” is meant any sequence of two or more amino acids, regardless of length, post-translation modification, or function. “Polypeptide”, “oligopeptide”, “peptide”, and “protein” are used interchangeably herein. The peptides or mimetics thereof of the invention are typically, though not universally, between 4 and 90 amino acids in length. In various embodiments a peptide of the invention may be less than 200 amino acids in length, less than 180 amino acids in length, less than 160 amino acids in length, less than 140 amino acids in length, less than 120 amino acids in length, less than 100 amino acids in length, less than 90 amino acids in length, less than 80 amino acids in length, less than 70 amino acids in length, less than 60 amino acids in length, less than 50 amino acids in length, less than 40 amino acids in length, less than 35 amino acids in length, less than 30 amino acids in length, less than 28 amino acids in length, less than 27 amino acids in length, 25 amino acids in length, less than 20 amino acids in length, less than 18 amino acids in length, less than 15 amino acids in length, less than 10 amino acids in length, or about 4 or 5 amino acids in length.

The term “salts” preferably refers to pharmaceutically acceptable, but it will be appreciated that non-pharmaceutically acceptable salts also fall within the scope of the present invention, since these are useful as intermediates in the preparation of pharmaceutically acceptable salts. Examples of pharmaceutically acceptable salts include salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulphuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulphonic, trihalomethanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

The term “solvates” refers to the interaction of amino acid analogues or peptides with water or common organic solvents. Such solvates are encompassed within the scope of the invention.

By “derivative” is meant any salt, hydrate, protected form, ester, amide, active metabolite, analogue, residue or any other compound which is not biologically or otherwise undesirable and induces the desired pharmacological and/or physiological effect. Preferably the derivative is pharmaceutically acceptable.

The term “tautomer” is used in its broadest sense to include amino acids and peptides which are capable of existing in a state of equilibrium between two isomeric forms. Such compounds may differ in the bond connecting two atoms or groups and the position of these atoms or groups in the compound.

The term “isomer” is used in its broadest sense and includes structural, geometric and stereoisomers. As the amino acids and peptides that may be synthesised by these techniques may have one or more chiral centres, they are capable of existing in enantiomeric forms. It is preferred that where the amino acid or peptide is present as a mixture of stereoisomers, the mixture is enriched in the preferred isomer.

The term “enriched” means that the mixture contains more of the preferred isomer than of the other isomer. Preferably, an enriched mixture comprises greater than 50% of the preferred isomer, where the preferred isomer gives the desired level of potency and selectivity. More preferably, an enriched mixture comprises at least 70%, 80%, 90%, 95%, 96%, 97%, 97.5%, 98% or 99% of the preferred isomer. The amino acid or peptide which is enriched in the preferred isomer can either be obtained via a stereospecific reaction, stereoselective reaction, isomeric enrichment via separation processes, or a combination of all three approaches.

The term “alkyl” refers to an optionally substituted monovalent alkyl group or an optionally substituted divalent alkylene group including straight chain and branched alkyl groups having from 1 to about 20 carbon atoms. Typically, the alkyl or alkylene group has from 1 to 15 carbons or, in some embodiments, from 1 to 8, 1 to 6, or 1 to 4 carbon atoms. Examples include methyl (Me), ethyl (Et), propyl (Pr), isopropyl (i-Pr), butyl (Bu), isobutyl (i-Bu), sec-butyl (s-Bu), tert-butyl (t-Bu), pentyl, neopentyl, hexyl and the like. Examples of straight chain alkyl or alkylene groups include methyl, methylene, ethyl, ethylene, n-propyl, n-trimethylene, n-butyl, n-tetramethylene, n-pentyl, n-pentamethylene, n-hexyl, n-hexamethylene, n-heptyl, n-heptamethylene, n-octyl and n-octamethylene groups. Examples of branched alkyl groups include, but are not limited to, isopropyl, iso-butyl, sec-butyl, tert-butyl, neopentyl, isopentyl, and 2,2-dimethylpropyl groups. Unless the context requires otherwise, the term “alkyl” also encompasses alkyl groups containing one less hydrogen atom such that the group is attached via two positions i.e. divalent. The alkyl or alkylene group may also be substituted and may include one or more substituents.

The term “heteroatom” refers to any atom other than carbon or hydrogen. The term “heteroatom” encompasses groups that are attached via two positions i.e. divalent. Preferably, the heteroatom is selected from the group consisting of oxygen, sulfur, nitrogen, phosphorus, silicon and boron. More preferably, the heteroatom is oxygen, sulfur, nitrogen or phosphorus. The heteroatom may be divalent and have its empty valences filled by hydrogen, oxygen, or an alkyl group provided that the heteroatom is not in a form which would poison the catalyst or affect its selectivity. Most free amines poison metathesis catalysts and therefore are preferably protected, provided as a quaternary amine salt or avoided during methathesis. For example, the divalent heteroatom may be selected from O, S(O), S(O)₂, SO₂NH, OS(O₂)O, SO₃, NH, N(R⁷), PO₄, HPO₂, P(R⁷)₂, OP(R⁷)₂, P(OR⁷)R⁷, P(OR⁷)₂, OP(OR⁷)R⁷, OP(OR)₂, P₂O₇, wherein each R⁷ is independently a substituted or unsubstituted C₁ to C₁₀ alkyl.

The term “alkyl group interrupted by one or more heteroatoms” refers to an alkyl or alkylene group as defined above, which is interrupted by one or more heteroatoms as defined above. Preferably, the one or more heteroatoms are selected from the group consisting of oxygen, sulfur, nitrogen, phosphorus, silicon and boron. More preferably, the heteroatom is oxygen, sulfur, nitrogen or phosphorus. The alkyl group may include any number of heteroatoms. Where more than one heteroatom is present, the heteroatoms may be adjacent one another or located between carbon atoms. Further, where more than one heteroatom is present, each heteroatom may be the same or different. The number of heteroatoms may be less than or equal to the number of carbon atoms present in the alkyl group. Preferably, the alkyl group has from 1 to about 20 carbon atoms, from 1 to 15 carbon atoms, from 1 to 8 carbon atoms, from 1 to 6 carbon atoms or from 1 to 4 carbon atoms and is interrupted by from 1 to about 20 heteroatoms, from 1 to 15 heteroatoms atoms, from 1 to 8 heteroatoms, from 1 to 6 heteroatoms or from 1 to 4 heteroatoms.

A “substituted” alkyl group or a “substituted” alkyl group interrupted by one or more heteroatoms has one or more of its hydrogen atoms replaced by non-hydrogen or non-carbon atoms. The term “substituted” or “substituent” as used herein refers to a group which may or may not be further substituted with 1, 2, 3, 4 or more groups, preferably 1, 2 or 3, more preferably 1 or 2 groups selected from the group consisting of C₁₋₆alkyl, C₂₋₆alkenyl, C₂₋₆alkynyl, C₃₋₈cycloalkyl, hydroxyl, oxo, C₁₋₆alkoxy, C₂₋₆alkenoxy, C₂₋₆alkynoxy, aryloxy, aralkyloxy, C₁₋₆alkoxyaryl, heterocyclyloxy, heterocyclylalkoxy, halo (such as F, Cl, Br, and I), C₁₋₆alkylhalo (such as CF₃ and CHF₂), C₁₋₆alkoxyhalo (such as OCF₃ and OCHF₂), carbonyls (oxo), carboxyl, esters, cyano, nitro, amino, substituted amino, disubstituted amino, nitrile (i.e. CN), acyl, ketones, amides, aminoacyl, substituted amides, disubstituted amides, N-oxides, hydrazines, hydrazides, hydrazones, azides, ureas, amidines, guanidines, enamines, imides, isocyanates, isothiocyanates, cyanates, thiocyanates, thiol, alkylthio, thioxo, sulfates, sulfonates, sulfinyl, substituted sulfinyl, sulfonyl, substituted sulfonyl, sulfonylamides, substituted sulfonamides, disubstituted sulfonamides, urethanes, oximes, hydroxylamines, alkoxyamines, aralkoxyamines, heterocyclyl and heteroaryl wherein each alkyl, alkenyl, alkynyl, cycloalkyl, aryl, heterocyclyl and heteroaryl and groups containing them may be further optionally substituted. Preferred optional substituents include C₁₋₄alkyl, C₂₋₄alkenyl, C₂₋₄alkynyl, C₃₋₆cycloalkyl, hydroxyl, oxo, C₁₋₄alkoxy, halo, C₁₋₄alkylhalo (such as CF₃ and CHF₂), C₁₋₄alkoxyhalo, carboxyl, esters, amino, substituted amino, disubstituted amino, ketones, amides, substituted amides, disubstituted amides, sulphonyl, substituted sulphonyl, aryl, arC₁₋₆alkyl, heterocycyl and heteroaryl, wherein each alkyl, alkenyl, alkynyl, cycloalkyl, aryl, heterocyclyl and heteroaryl and the group containing them may be further optionally substituted. Such substituents should not be groups that poison the metathesis catalyst or affect its selectivity. Most free amines poison metathesis catalysts and therefore are preferably protected, provided as a quaternary amine salt or avoided during methathesis. Preferably, the substituents may be selected from esters, carbonyls (oxo) including aldehydes and ketones, carboxyls, amides, nitriles and alcohols. As another example, an alkyl group may be substituted with one or more halogens, such as fluorine.

Dicarba Bridge

The method of the present invention relates to the formation of at least one dicarba bridge between an amino acid containing a metathesisable group and another compound containing a complementary metathesisable group. The present invention also relates to peptides containing dicarba bridges which are formed by metathesis of a reactable peptide containing a metathesisable group and a compound containing a metathesisable group, or by metathesis of two metathesisable groups contained in a single reactable peptide.

The dicarba bridge may be formed between two separate peptide chains to form an interchain dicarba bridge, or it may form a bridge between two points in a single peptide chain so as to form an intra-chain dicarba bridge, otherwise known as a ring.

In some instances it may be difficult to form dicarba bridges due to steric hinderance, aggregation and/or the need to bring the reactable (metathesisable) groups together. However, the use of alternating solid phase peptide synthesis and other strategies, as described herein, such as microwave reaction conditions and the use of turn-inducing groups and removable tethers may be employed to enhance the catalysis steps.

The term “dicarba bridge” is used broadly, unless the context indicates otherwise, to refer to a bridging group that includes at least one of the groups selected from —C—C— and —C═C—. This means that the dicarba bridge could be wholly or partly composed of the groups —C—C— and —C═C—. The atoms directly attached to the carbon atoms of the dicarba bridge sequence are carbon. Where possible, further or alternative reactions may be performed to introduce substituents other than hydrogen onto the carbon atoms of the dicarba sequence of the dicarba bridge.

The term “unsaturated dicarba bridge” is used broadly, unless the context indicates otherwise, to refer to a bridging group that includes at least an unsaturated alkene (—C═C. This means that the dicarba bridge could be wholly or partly composed of the group —C═C—.

The term “alkene-containing dicarba bridge” or “unsaturated hydrogen dicarba bridge” as used herein refers to dicarba bridges which contain the group —CH═CH—. This means that the alkene-containing dicarba bridge could be wholly or partly composed of the group —C═C—. The alkene-containing dicarba bridge (—C═C—) may be cis or trans-geometry.

The term “saturated dicarba bridge” is used broadly, unless the context indicates otherwise, to refer to a bridging group that includes at least a saturated alkane containing dicarba bridge (—C—C—). This means that the dicarba bridge could be wholly or partly composed of the groups —C—C—.

In addition to the unsaturated or saturated dicarba sequence formed between the amino acid containing a dicarba bridge and the compound containing a complementary metathesisable group, the dicarba bridge may include any other series of atoms, typically selected from C, N, O, S, and P, with the proviso that the nitrogen atoms present in the compound during metathesis are not free amines (protected amines, such as carbamates, and salts are acceptable).

Where the terms “alkene-containing” “unsaturated”, “alkane-containing” or “saturated” are not specified, the term “dicarba bridge” is taken to refer to a bridging group that includes at least one of the groups selected from a saturated dicarba bridge (—C—C—) or an unsaturated alkene-containing dicarba bridge (—C═C—), as described above.

Amino Acids and Reactable Peptides Containing a Metathesisable Group

An amino acid containing a metathesisable group is one of the starting materials used in the synthesis of amino acid analogues.

The amino acid may be in the form of a free compound, or in the form of a salt, solvate, derivative, isomer or tautomer thereof.

The amino acid may be an L-amino acid or a D-amino acid, or as a mixture of any ratio of stereoisomers.

Reactable peptides containing metathesisable groups are one of the starting materials used in the methods for the synthesis of a peptide or peptides containing a dicarba bridge. The term “reactable peptide” or “reactable peptides” is intended to refer to a peptide containing one or more metathesisable groups or peptides which contain one or more metathesisable groups between them.

The reactable peptide may be in the form of a free compound, or in the form of a salt, solvate, derivative, isomer or tautomer thereof.

The reactable peptide may be composed or L-amino acids or a D-amino acids, or as a mixture of any ratio of stereoisomers.

The term “metathesisable group” is used broadly, unless the context indicates otherwise, to refer to at least an alkene moiety. The alkene moiety may be present as an E- or Z-configured alkene, or a mixture of any ratio of geometric isomers. Preferably, the alkene-containing dicarba bridge is enriched in the preferred isomer.

It is noted that a pair of complementary alkene-containing metathesisable groups need not be identical. As one example, an allylglycine residue can be metathesised with a crotylglycine residue to generate a new dicarba bridge. The term “complementary” is used to indicate that the pair of alkene-containing metathesisable groups are not necessarily identical, but are merely complementary in the sense that metathesis can take place between the two alkene-containing groups.

In one embodiment, the alkene-containing metathesisable group has the general formula (IV) as shown below.

In another embodiment, the alkene-containing metathesisable group is covalently attached to an amino acid and the amino acid having the metathesisable group is a compound of formula (VI) as shown below.

In one embodiment, the alkene-containing metathesisable group or groups of the reactable peptide are of the general formula (IV) as shown below.

In another embodiment, the alkene-containing metathesisable group is covalently attached to an amino acid of the reactable peptide, preferably being located on the amino group or on the side chain of an amino acid of the reactable peptide. In one embodiment, the reactable peptide containing the metathesisable group or groups is a peptide containing at least one compound of formula (VI¹) as shown below.

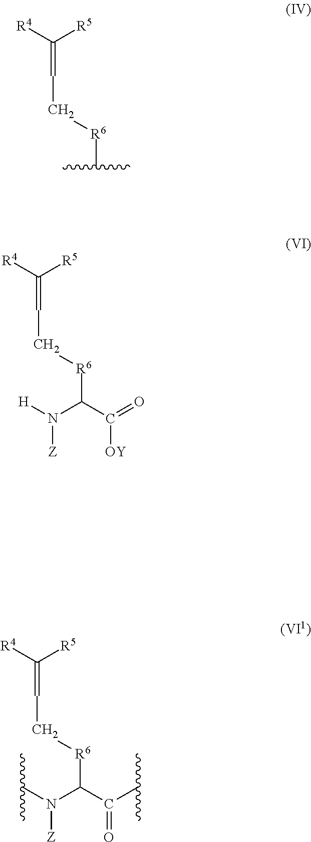

The groups R⁴, R⁵ and R⁶ should not be a group which poisons the metathesis catalyst.

In one embodiment, the groups R⁴ and R⁵ are independently selected from H and substituted or unsubstituted C₁ to C₄ alkyl. When R⁴ or R⁵ are substituted alkyl, the substituents are preferably one or more halogens, such as, for example, fluorine.

During the metathesis reaction, a by-product containing an alkene bond substituted with the groups R⁴ and R⁵ is produced. Preferably, the groups R⁴ and R⁵ are such that the resulting by-product is gaseous, and is eliminated from the reaction mixture. For example, in formula (IV) when R⁴ and R⁵ are hydrogen and the corresponding groups on the complementary metathesisable group are also hydrogen, the by-product is ethylene, which evaporates from the reaction mixture to leave the reaction product. Preferably, the groups R⁴ and R⁵ are each independently either H or methyl. It will however be appreciated that techniques for the separation of a non-gaseous by-product from the reaction mixture would also be known by a person skilled in the art.

The group R⁶ is either present or absent. When R⁶ is present it is a linker between the metathesisable group and, for example, the amino acid reactant or backbone. When R⁶ is absent, the divalent methylene group adjacent the alkene double bond in formula (IV) is the linker.

The group R⁶, when present, is selected from a heteroatom, a substituted or unsubstituted C₁ to C₂₀ alkyl, and a substituted or unsubstituted C₁ to C₂₀ alkyl interrupted by one or more heteroatoms.

When the group R⁶ is a heteroatom, the heteroatom is preferably oxygen, sulfur, nitrogen or phosphorus. When the heteroatom is a nitrogen, it is preferably protected, provided as a quaternary amine salt or avoided during methathesis. The heteroatom may be selected from the group consisting of O, S(O), S(O)₂, SO₂NH, OS(O₂)O, NH, N(R⁷), PO₄, and P(R⁷)₂, wherein each R⁷ is independently a substituted or unsubstituted C₁ to C₁₀ alkyl.

In one embodiment, the group R⁶ is a substituted or unsubstituted alkyl group having from 1 to about 20 carbon atoms, from 1 to 15 carbon atoms, from 1 to 8 carbon atoms, from 1 to 6 carbon atoms or from 1 to 4 carbon atoms. Preferably, the group R⁶ has from 1 to 8 carbon atoms. Most preferably, R⁶ is methyl or ethyl.

When the group R⁶ is a substituted or unsubstituted alkyl interrupted by one or more heteroatoms, preferably, the alkyl group has from 1 to about 20 carbon atoms, from 1 to 15 carbon atoms, from 1 to 8 carbon atoms, from 1 to 6 carbon atoms or from 1 to 4 carbon atoms and is interrupted by from 1 to about 20 heteroatoms, from 1 to 15 heteroatoms atoms, from 1 to 8 heteroatoms, from 1 to 6 heteroatoms or from 1 to 4 heteroatoms. The heteroatoms may be selected from the group consisting of N, O, S, P and mixtures thereof. When the heteroatom is a nitrogen, it is preferably protected, provided as a quaternary amine salt or avoided during methathesis. More preferably, R⁶ is an alkyl group having from 1 to 8 carbon atoms that is interrupted by from 1 to 3 heteroatoms.

When R⁶ is a substituted alkyl or substituted alkyl interrupted by one or more heteroatoms, the substituents are groups that do not poison the metathesis catalyst or affect its selectivity. Preferably, the substituents may be selected from esters, carbonyls (oxo) including aldehydes and ketones, carboxyls, amides, nitriles and alcohols.

The group Z in formula (VI) or (VI¹) is selected from H, a salt and a protecting group. When Z is a protecting group, it may be selected from the group consisting of 9-fluorenylmethyl carbamate (Fmoc), 2,2,2-trichloroethyl carbamate (Troc), t-butyl carbamate (Boc), allyl carbamate (Alloc), 2-trimethylsilylethyl (Teoc) and benzyl carbamate (Cbz). Preferably the group Z is Fmoc.

The group Y in formula (VI) is selected from H and a protecting group. When Y is a protecting group, it may be an ester such as an alkyl ester, for example, methyl ester, ethyl ester, t-Bu ester or a benzyl ester.

In one embodiment, the reactable peptide containing a metathesisable group or groups is a peptide containing at least one amino acid residue selected from optionally protected allylglycine, optionally protected crotylglycine, optionally protected butynylglycine and optionally protected butenylglycine. Preferably, the reactable peptide containing a metathesisable group or groups is a peptide containing at least one amino acid residue selected from optionally protected allylglycine, optionally protected butynylglycine and optionally protected butenylglycine.

The reactivity of alkenes towards homodimerisation during metathesis has been categorised into four classes—Type I through IV. Type I alkenes are the most reactive and are characterised by sterically unhindered and electron-rich olefins such as allyl- (A) and crotyl-glycine (B). Increasing steric hindrance and decreasing electron density about the olefin results in slower homodimerisation and sees these alkenes categorised in Types II through IV. These include residues such as prenylglycine (C) and the extended acrylate (D). These glycine derivatives are shown below.

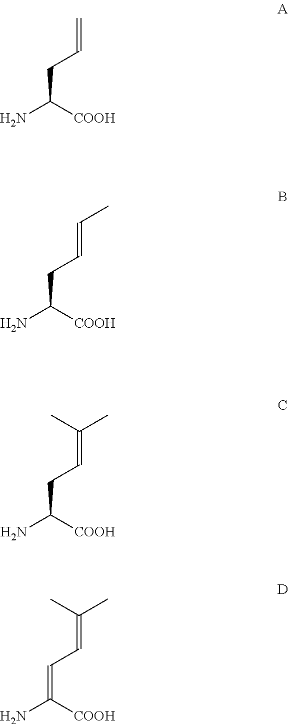

In one embodiment, the amino acid having the metathesisable group is selected from optionally protected allylglycine, optionally protected crotylglycine, optionally protected butynylglycine or optionally protected butenylglycine. Preferably, the amino acid having the metathesisable group is selected from optionally protected allylglycine, optionally protected butynylglycine or optionally protected butenylglycine.

Compound Containing a Complementary Metathesisable Group

In the method for the synthesis of an amino acid analogue or a salt, solvate, derivative, isomer or tautomer thereof, an amino acid containing a metathesisable group as described above is reacted with a compound containing a complementary metathesisable group of formula (I) or (II):

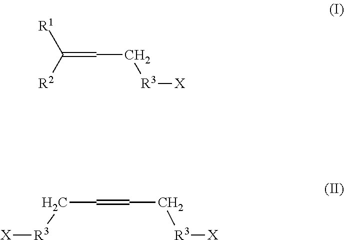

to form a dicarba bridge between the amino acid containing a metathesisable group and the compound containing a complementary metathesisable group. Preferably, the metathesis is not self-metathesis in that the reactants provided are not the same. In other words, the amino acid containing a metathesisable group and the compound of formula (I) are not identical compounds.

The groups R¹, R² and R³ should not be a group which poisons the metathesis catalyst.

The groups R¹ and R² are independently selected from H and substituted or unsubstituted C₁ to C₄ alkyl. Preferably, when R¹ or R² are substituted alkyl, the substituent at the carbon alpha to the alkene double bond is not oxo. When R¹ or R² are substituted alkyl, the substituents are preferably one or more halogens, such as, for example, fluorine.

As described above, the metathesis reaction generates a by-product containing an alkene bond substituted at one end with the groups R⁴ and R⁵ from the metathesisable group of the amino acid of formula (IV) and, for example, the groups R¹ and R² of formula (I) at the other end. Accordingly, it is preferable that the groups R¹, R², R⁴ and R⁵ are such that the resulting by-product is gaseous, and is eliminated from the reaction mixture. Preferably, the groups R¹ and R² are independently selected from H and methyl. It will however be appreciated that techniques for the separation of a non-gaseous by-product from the reaction mixture would also be known by a person skilled in the art.

The group R³ is either present or absent. When R³ is present it is a linker between the metathesisable group and the group X. When R³ is absent, the divalent methylene group adjacent the alkene double bond in formula (I) or (II), or the divalent methylene group adjacent the alkyne double bond in formula (III) is the linker between the metathesisable group and the group X.

The group R³, when present, may be selected from a heteroatom, a substituted or unsubstituted C₁ to C₂₀ alkyl, and a substituted or unsubstituted C₁ to C₂₀ alkyl interrupted by one or more heteroatoms.

When the group R³ is a heteroatom, the heteroatom is preferably oxygen, sulfur, nitrogen or phosphorus. When the heteroatom is a nitrogen, it is preferably protected, provided as a quaternary amine salt or avoided during methathesis. The heteroatom may be selected from the group consisting of O, S(O), S(O)₂, SO₂NH, OS(O₂)O, NH, N(R⁷), PO₄, and P(R⁷)₂, wherein each R⁷ is independently a substituted or unsubstituted C₁ to C₁₀ alkyl.

When the group R³ is a substituted or unsubstituted alkyl, it is preferably an alkyl group having from 1 to about 20 carbon atoms, from 1 to 15 carbon atoms, from 1 to 8 carbon atoms, from 1 to 6 carbon atoms or from 1 to 4 carbon atoms. More preferably, R³ is an alkyl group having from 1 to 8 carbon atoms.

When the group R³ is a substituted or unsubstituted alkyl interrupted by one or more heteroatoms, preferably, the alkyl group has from 1 to about 20 carbon atoms, from 1 to 15 carbon atoms, from 1 to 8 carbon atoms, from 1 to 6 carbon atoms or from 1 to 4 carbon atoms and is interrupted by from 1 to about 20 heteroatoms, from 1 to 15 heteroatoms atoms, from 1 to 8 heteroatoms, from 1 to 6 heteroatoms or from 1 to 4 heteroatoms. The heteroatoms may be selected from the group consisting of N, O, S, P and mixtures thereof. When the heteroatom is a nitrogen, it is preferably protected, provided as a quaternary amine salt or avoided during methathesis. More preferably, R³ is an alkyl group having from 1 to 8 carbon atoms that is interrupted by from 1 to 3 heteroatoms.

When R³ is a substituted alkyl or substituted alkyl interrupted by one or more heteroatoms, the substituents are groups that do not poison the metathesis catalyst or affect its selectivity. Preferably, the substituents may be selected from esters, carbonyls (oxo) including aldehydes and ketones, carboxyls, amides, nitriles and alcohols.

Each X is independently either H or an effector molecule.

In the method for the synthesis of a peptide or peptides containing a dicarba bridge or a salt, solvate, derivative, isomer or tautomer thereof, a reactable peptide containing a metathesisable group as described above is reacted with a compound containing a complementary metathesisable group of formula (I′) or (II′):

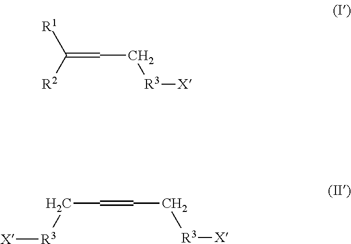

The compounds of formula (I′), and (II′) contain the group X′. The other groups of the compounds of formula (I′) and (II′), namely R¹, R² and R³ are the same as the groups R¹, R² and R³ as defined in relation to the compounds of formula (I) and (II).

X′ is selected from H, an effector molecule, an amino acid and a peptide. When X′ is an amino acid or a peptide, the method can be used to create a dicarba bridge between an amino acid or a reactable peptide containing a metathesisable group and a compound of formula (I) and (II) in which X′ is an amino acid or a peptide. For example, the dicarba bridge may be formed between two separate peptide chains (the reactable peptide and the compound in which X′ is a peptide) to form an interchain dicarba bridge. This strategy can be used to replace naturally occurring disulfide bridges present between peptide subunits with dicarba bridges.

When the compound containing the complementary metathesisable group has the formula (II), preferably, each of the groups R³ are the same and each X is the same. In this embodiment, the compound of formula (II) is symmetrical. In theory, one molecule of the symmetrical compound of formula (II) will react with two molecules of the amino acid containing a metathesisable group. However, it will be appreciated that other ratios of the reactants may be used. For example, the use of a molar excess of the compound of formula (II) may be used to drive the conversion of the reactants into the amino acid analogue.

Effector Molecule

The amino acid analogue or salt, solvate, derivative, isomer or tautomer thereof of the invention may be conjugated to an effector molecule. This product may be formed by metathesis between an amino acid containing a metathesisable group and a compound of formula (I) or (II) in which the group X is an effector molecule.

The term “effector molecule” is used broadly, unless the context indicates otherwise, to refer to any molecule derivatised in a way to enable cross metathesis with a second molecule. Preferably, the effector molecule is a chemical moiety capable of providing an effect in a biological system. Preferably, the effector molecule is not an amino acid or salt thereof.

In one embodiment, the effector molecule is a chemical moiety capable of providing a therapeutic effect. In this embodiment, the chemical moiety is a therapeutic agent, or a prodrug which is converted into the therapeutic agent in vivo. Conjugation of the therapeutic agent or prodrug to an amino acid analogue or into a peptide may improve the absorption, and/or biological availability of the therapeutic agent or prodrug. Incorporation of an amino acid analogue conjugated to a therapeutic agent or prodrug into a peptide may also improve the absorption, and/or biological availability of the therapeutic agent or prodrug, or permit targeted delivery of the therapeutic agent or prodrug to a particular site.

In another embodiment, the effector molecule is a chemical moiety capable of providing a detectable effect, which enables the identification or location of the effector molecule. In this embodiment, the chemical moiety is a labelling agent or tag. Conjugation of the labelling agent or tag to an amino acid analogue using the method of the present invention is a simple means of incorporating such a labelling agent or tag into an amino acid. Furthermore, peptides may be prepared using the amino acid analogue conjugated to the tag or labelling agent, with unlimited flexibility in relation to the location of the tag or labelling agent in the peptide. Detection of the labelled peptide can, for example, allow determination of the distribution of the peptide within the body, the rate and/or method by which the peptide is metabolised in the body, and/or determination of how the peptide is excreted from the body. The labelled or tagged amino acid or peptide may also be used in a variety of other applications, such as for example, receptor-ligand binding studies, protease inhibitor screening, signal transduction research and immunoassays. Examples of effector molecules which may be conjugated to the amino acid analogue or peptide include molecules comprising fluorescent labels (e.g. fluorescein), dyes, chemiluminescent compounds, biotins, other haptens such as digoxigenin and dinitrophenol (DNP), carbohydrates, lipids, chelating agents, nanoparticles, enzymes and radioisotopes.

The tag may, for example, be any known tag used in the detection of amino acids or peptides. The labelling agent may be any known labelling agent used in the detection of amino acids or peptides, for example, the labelling agent may be a chelate capable of binding a radioactive isotope for radio labelling of the amino acid analogue or amino acid analogue containing peptide. In another aspect, the label may be a radiopharmaceutical. The radiopharmaceuticals may be for diagnostic or interventional purposes.

In one embodiment, the effector molecule comprises a lipid. The method of the present invention can be applied to the synthesis of amino acid analogues, or peptides comprising amino acid analogues, having effector molecules comprising extended fatty acid chains, both branched and unbranched. For example, effector molecules comprising C₄, C₅, C₆, C₇, C₈, C₉ and C₁₀ or greater fatty acid chains can be conjugated to the amino acid analogues, or peptides comprising amino acid analogues, using the method of the present invention. In one embodiment, the effector molecule comprises a fatty acid chain comprising at least 8, 9, 10, 11 or 12 carbons.

The conjugation of hydrophobic moieties such as fatty acid chains may be used to enhance cell membrane integration or penetration and/or to improve oral bioavailability.

Metathesis

Metathesis is a powerful synthetic tool that enables the synthesis of carbon-carbon bonds via a transition metal-catalysed transformation of alkyl-unsaturated reactants. The construction of dicarba analogues of complex peptides, however, presents more of a synthetic challenge.

The use of uniform metathesis substrates leads to a statistical product distribution and therefore metathesis selectivity is severely compromised. For example, homodimerisation of equivalent olefins A and B in the absence of selectivity results in a statistical mixture of three products (as shown below). The yield of desired products (A-A and B-B) is not more than 50% in the absence of selection. In order to exclusively form the target A-B product, selective metathesis strategies must be employed to avoid the formation of the A-A and B-B homodimers.

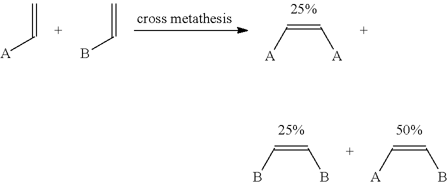

Cross-metathesis (CM) is a type of metathesis reaction involving the formation of a new bond across two unblocked, reactive metathesisable groups, to form a new bridge between the two reactive metathesisable groups. For example, using cross-metathesis, a dicarba analogue of a peptide having an intermolecular bridge results from formation of a dicarba bridge between two reactive peptides each containing a complementary metathesisable group.

Ring-closing metathesis (RCM) is a type of metathesis reaction where the two reactive metathesisable groups are located within one peptide chain so as to form an intramolecular bridge, or ring. For example, ring-closing metathesis involves the formation of a dicarba bridge between two complementary metathesisable groups located on a single peptide chain to produce a dicarba analogue of a peptide having an intrachain bridge.

It is preferred that at least one reactable peptide is provided on a solid support. The types of solid supports that may be used are described below.

The use of a solid support provides a number of advantages. Firstly, the combination of peptide synthesis and catalysis using a single solid support is highly efficient. In addition, the catalysts used may be homogeneous catalysts, such as those used to affect metathesis and hydrogenation. The catalysts can be exposed to a resin bound peptide and simply separated from the product peptide via filtration of the resin-peptide from the reaction solution. This eliminates and/or minimises metal-contamination of the product and aids the separation of the product peptide from solution phase by-products and/or impurities. Furthermore, protecting groups for reactive sidechains which are commonly employed in SPPS protocols are also tolerated by organotransition metal catalysts and hence catalysis can conveniently be performed immediately after SPPS.

Tethering a peptide sequence to a solid support can also promote RCM. A pseudo-dilution effect operates on resin to promote RCM over otherwise competing CM reactions. Hence high dilution is not required for the promotion of RCM conversion.

Alkene Metathesis

Alkene metathesis provides a versatile method for the cleavage and formation of C═C bonds, and involves a mutual intermolecular exchange of alkylidene fragments between two alkene groups.

In metathesis reactions, the redistribution can result in three main outcomes shown below: (A) ring-opening metathesis (ROM) which is sometimes followed by polymerization of the diene (ROMP); (B) ring-closing metathesis (RCM); and (C) cross metathesis (CM). Of particular interest to the present invention is cross metathesis.

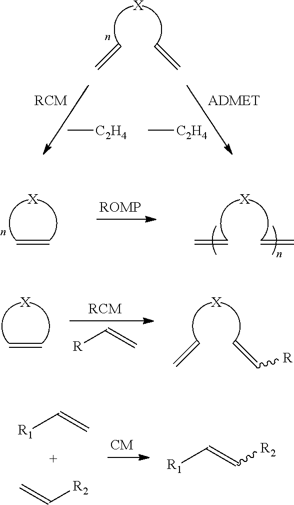

When a pair of complementary metathesisable groups are incorporated into two separate compounds and subjected to metathesis conditions, an intermolecular cross-metathesis (CM) reaction will form a dicarba bridge between the compounds. If however, the pair of alkene-containing metathesisable groups are incorporated into the same compound, such as the primary sequence of a single peptide, and the peptide is subjected to metathesis conditions, an intramolecular ring-closing metathesis reaction (RCM) will result in the formation of a cyclic compound, such as a cyclic peptide.

In the methods of the present invention, the alkene-containing dicarba bridge that is formed by alkene metathesis is subsequently reduced. In a preferred embodiment, a single catalyst is used both to form the dicarba bridge and reduce the dicarba bridge to the corresponding unsaturated dicarba bridge, i.e. a recycled metathesis catalyst is employed in a tandem metathesis-reduction method.

Catalysts which may be used to perform alkene metathesis in the method of the present invention are those catalysts which are selective for the alkene-containing metathesisable groups, while not interfering with the functional groups present in the amino acids and the complementary metathesisable group containing compound between which the alkene-containing dicarba bridge is formed. There are many metathesis catalysts known in the art. Examples of suitable catalysts include those described in Grubbs, R. H., Vougioukalakis, G. C. Chem. Rev., 2010, 110, 1746-1787; Tiede, S., Berger, A., Schlesiger, D., Rost, D., Luhl, A., Blechert, S., Angew. Chem. Int. Ed., 2010, 49, 1-5; and Samojlowicz, C., Bieniek, m., Grela, K. Chem. Rev., 2009, 109, 3708-3742, which are incorporated herein by reference. Preferably, the catalyst used for alkene metathesis is a homogeneous catalyst, such as a ruthenium-based alkene metathesis catalyst.

Many alkene metathesis catalysts are now commercially available or easily synthesised in the laboratory. While early catalysts were poorly defined, lacked functional group tolerance and were highly moisture and oxygen sensitive, later generation catalysts have largely overcome these initial problems. Currently used Ru-based catalysts, for example Grubbs' first and second generation catalysts and the Hoveyda-Grubbs analogues, are robust, display high functional group tolerance and have tunable reactivity under mild experimental conditions. Despite their differing substitution around the core Ru centre, all of the catalysts cycle through an active ruthenium alkylidene species. The variation around the reactive core however, plays an important role in mediating initiation, propagation and substrate specificity.

In the tandem metathesis-reduction method, the same reagent or catalyst is used to catalyse the metathesis step and the reduction step. This reagent is referred to herein as a “recycled metathesis catalyst”, which is a decomposition of the metathesis catalyst that is generated during the metathesis reaction.

Examples of suitable catalysts which may be used in tandem-metathesis include ruthenium-alkylidene catalysts. These catalysts are composed of a ruthenium alkylidene along with two anionic and two neutral ligands. The anionic ligands may be halogens such as chlorides, monodentate and bidentate aryloxides, N,O-, P,O- and O,O-bidentate ligands, carboxylates and (allkyl)sulfonates. The neutral ligands may be phosphine ligands such as tricyclohexyl phosphines, heterocyclic carbene ligands including N-heterocyclic carbene ligands (such as symmetrical or unsymmetrical imidazol-2-ylidenes, triazol-5-ylidenes, tetrahydropyrimidine-2-ylidenes, four-membered ring diaminocarbenes, cyclic (alkyl)(amino)carbenes and thiazol-2-ylidenes), chelating alkoxybenzylidene ligands, chelating thioether ligands, chelating sulfoxide benzylidene ligands, mono- and bis(pyridine)-coordinated catalysts, chelating quinolin-ylidenes, or alkylidene ligands (such as bidentate alkylidenes chelated through imine donors or 14-electron phosphonium alkylidenes). Preferably, the catalyst is a non-phosphine ruthenium alkylidene catalyst. Examples of suitable non-phosphine ruthenium-alkylidene catalysts include the first and second generation Grubbs catalysts, and the first and second generation Grubbs-Hoveyda catalysts such as, for example, those shown below. Most preferably, the catalyst is a Hoveyda-Grubbs catalyst.

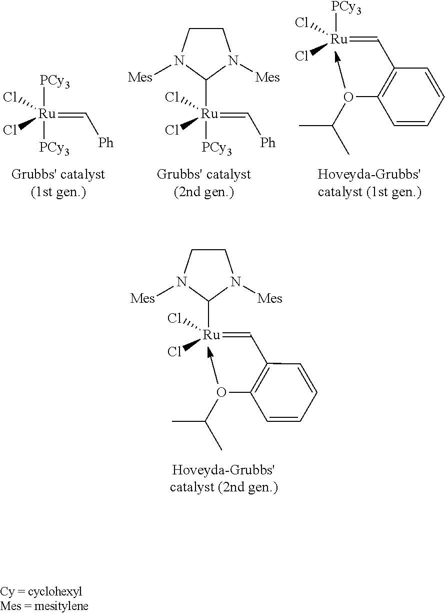

Advantageously, when the reaction employs a Ru-based metathesis catalysts, unnecessary functional group protection and deprotection steps may be eliminated from the reaction process. As one example, the carboxylic acid functionality is well tolerated by Ru-based metathesis catalysts, and as such, carboxylic acids on either of the reactants do not require protection, and so the protection and deprotection steps may be eliminated from the reaction process.

One problem which may be associated with metathesis processes is the formation of by-products by concomitant olefin isomerisation and secondary metathesis processes. The use of reactants which produce a tri-substituted alkene product (as shown below) can ensure minimal olefin isomerisation as the trisubstituted alkene is the most stable product. However, in the method of the present invention, the reactants include a methylene group at the position adjacent the alkene double bond, and as such there is not one most stable product. In this case, the formation of by-products by concomitant olefin isomerisation and secondary metathesis processes may occur (as shown below).

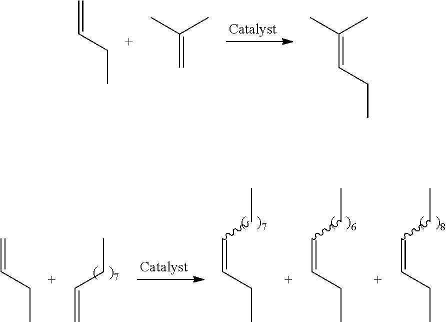

Under certain reaction conditions the formation of the undesired homologues may be minimized. As one example, conducting the reaction at room temperature and/or conducting the reaction with a continuous flow of nitrogen through the head space, can optimise production of the target cross product and increase the conversion of starting material to the target product.

Reaction Conditions for Metathesis

The metathesis reaction between an amino acid having a metathesisable group and a compound having a complementary metathesisable group may be performed in any solvent which provides good catalytic turnover and good conversion of the starting materials into the amino acid analogue.

The solvent may be any polar solvent which does not adversely affect the metathesis and hydrogenation catalyst(s) or the yield of the amino acid analogue. Preferably, the solvent is a polar aprotic solvent, such as dichloromethane or ethyl acetate.

The metathesis may be performed at any temperature ranging from reflux to room temperature. Preferably the metathesis reaction is conducted at ambient or room temperature. It has been found that by conducting the metathesis reaction at ambient temperature the formation of undesired homologues (such as by-products produced by concomitant isomerisation and secondary metathesis products) may be minimized, and production of the target cross product may be optimized.

The stoichiometry of the metathesis reaction may be adjusted as necessary. In some embodiments, the metathesis reaction will be conducted with a molar ratio of the compound of formula (I) or (II) to the amino acid containing a metathesisable group of from 1:1 up to 20:1, from 1:1 up to 10:1, from 1:1 up to 5:1, from 5:1 up to 10:1, at about 1:1, 2:1, 5:1 or 10:1.

The metathesis may be performed under continuous flow of inert gas (such as nitrogen), for example, through the head space of the reaction vessel. It has been found that conducting the metathesis reaction under a continuous flow of nitrogen through the head space, can optimise production of the target cross product and increase the conversion of starting material to the target product.

Other additives may also be added to the metathesis reaction. For example, the reaction may be conducted in the presence of molecular sieves.

Reduction of an Unsaturated Dicarba Bridge

The product of the cross-metathesis reaction is an amino acid analogue with an unsaturated dicarba bridge. In some instances, the dicarba bridge may need to assume a particular conformation in order to serve as a suitable peptidomimetic. It may therefore be advantageous for the dicarba bridge to adopt a particular geometry. The dicarba bridge conformation will change if the dicarba bridge is saturated compared to an unsaturated dicarba bridge.

If the target amino acid analogue is to contain a saturated dicarba bridge, the process further comprises the step of subjecting the unsaturated dicarba bridge to reduction. The reduction may be performed by hydrogenation, using a catalyst or by, for example, hydrosilylation and protodesilylation.

In the tandem metathesis-reduction method, the same reagent or catalyst is used to catalyse the metathesis step and the reduction step.

Hydrogenation

Hydrogenation of the unsaturated dicarba bridge can be performed with any known hydrogenation catalyst. Examples of suitable catalysts include those described in March, J. Advanced Organic Chemistry: Reactions, Mechanisms and Structure. 1992, pages 771 to 780, and in Ojima, I. Catalytic Asymmetric Synthesis; Wiley-VCH: New York, 2000; Second Edition, Chapter 1, 1-110, incorporated herein by reference. Suitable hydrogenation catalysts are chemoselective for unblocked non-conjugated carbon-carbon double or triple bonds.

Suitable hydrogenation catalysts may be either insoluble in the reaction medium (heterogeneous catalysts) or soluble in the reaction medium (homogeneous catalysts). Examples of suitable heterogeneous catalysts include Raney nickel, palladium-on-charcoal (Pd/C) and platinum oxide. Examples of suitable homogeneous catalysts include Wilkinson's catalyst, other Rh(I) phosphine complexes, and Ru(II) phosphine complexes.

If the target compound is to include a saturated alkane-containing dicarba bridge, the hydrogenation is performed with a catalyst that is chemoselective for non-conjugated carbon-carbon double bonds as distinct from other double bonds such as carbon-oxygen double bonds in carbonyl groups, and carboxylic acids.

Any catalyst which is chemoselective for non-conjugated carbon-carbon double bonds may be used. Examples of hydrogenation catalysts capable of reducing an alkene bond to an alkane bond include palladium-on-charcoal (Pd/C), platinum oxide, and Raney nickel. Hydrogenation catalysts which are suitable for reducing an alkene bond to an alkane bond also include asymmetric hydrogenation catalysts.

Although the use of an asymmetric hydrogenation catalyst is not necessary in the hydrogenation of the alkene-containing dicarba bridges, asymmetric hydrogenation catalysts can nevertheless be used. Suitable catalysts are well known in the art, and include the range of catalysts described for this purpose in Ojima, I. Catalytic Asymmetric Synthesis; Wiley-VCH: New York, 2000; Second Edition, Chapter 1, 1-110, the entirety of which is incorporated by reference. New catalysts having such properties are developed from time to time, and these may also be used. Further examples of suitable asymmetric hydrogenation catalysts are the chiral phosphine catalysts, including chiral phospholane Rh(I) catalysts. Catalysts in this class are described in U.S. Pat. No. 5,856,525. Such homogenous hydrogenation catalysts are tolerant of sulfide, and disulfide bonds, so that the presence of disulfide bonds and the like will not interfere with the synthetic strategy.

In the tandem metathesis-reduction method, the reagent which has been used to catalyse the metathesis reaction may also catalyse the reduction without the need for work-up of the metathesis product prior to reduction. This reagent is referred to herein as a “recycled metathesis catalyst”, and the process involving a metathesis step immediately followed by a reduction step via a single catalyst is herein referred to as “tandem metathesis-hydrogenation”. While not wishing to be bound by theory, exposure of the recycled metathesis catalyst to hydrogen is thought to afford a hydride complex (a decomposition product of the metathesis catalyst that is generated during the metathesis reaction) which is capable of functioning as a hydrogenation catalyst, however, the nature of the complex responsible for hydrogenation is not clear. However, the recycled catalysts can be left in air for several days and still generate an active hydrogenation catalyst on exposure to a hydrogen atmosphere.

Catalysts which may be used in tandem-metathesis include Ru-alkylidene catalysts. These catalysts are composed of a ruthenium alkylidene along with two anionic and two neutral ligands. The anionic ligands may be halogens such as chlorides, monodentate and bidentate aryloxides, N,O-, P,O- and O,O-bidentate ligands, carboxylates and (allkyl)sulfonates. The neutral ligands may be phosphine ligands such as tricyclohexyl phosphines, heterocyclic carbene ligands including N-heterocyclic carbene ligands (such as symmetrical or unsymmetrical imidazol-2-ylidenes, triazol-5-ylidenes, tetrahydropyrimidine-2-ylidenes, four-membered ring diaminocarbenes, cyclic (alkyl)(amino)carbenes and thiazol-2-ylidenes), chelating alkoxybenzylidene ligands, chelating thioether ligands, chelating sulfoxide benzylidene ligands, mono- and bis(pyridine)-coordinated catalysts, chelating quinolin-ylidenes, or alkylidene ligands (such as bidentate alkylidenes chelated through imine donors or 14-electron phosphonium alkylidenes). Preferably, the catalyst is a non-phosphine ruthenium alkylidene catalyst. Examples of suitable non-phosphine ruthenium-alkylidene catalysts include the first and second generation Grubbs catalysts, and the first and second generation Grubbs-Hoveyda catalysts as shown below. Most preferably, the catalyst is a Hoveyda-Grubbs catalyst.

The controlled reduction of C═C in organic compounds is an important synthetic transformation and many catalysts are available to achieve this end.

Where the dicarba bridge of the peptide or peptides is an alkene-containing dicarba bridge, the alkene-containing group of the bridge may be present as a mixture of any ratio of geometric isomers (e.g. E- or Z-configured alkenes), or as an enriched geometric isomer. As defined above, “enriched” means that the mixture contains more of the preferred isomer than of the other isomer.

Hydrogenation of the unsaturated dicarba bridge can be conducted at any temperature, such as room temperature or at elevated temperature. The reaction is typically conducted at elevated pressure, although if slower reaction times can be tolerated, the reaction can be performed at atmospheric pressure.

Hydrogenation of the unsaturated dicarba bridge can be by homogeneous or heterogeneous reaction. Homogeneous hydrogenation is used in its broadest sense to refer to catalytic hydrogenations conducted in one phase such as a liquid phase, where the liquid phase contains the substrate molecule/s and solvent. More than one solvent, such as organic/aqueous solvent combinations, or fluorous solvent combinations, non-aqueous ionic pairs, supercritical fluids, or systems with soluble polymers may also be employed. This is distinct from heterogeneous reactions, which involve more than one phase—as in the case of hydrogenations performed with solid-supported catalysts in a liquid reaction medium.

Reaction Conditions for Reduction

The reduction of the unsaturated dicarba bridge may be performed in any solvent which provides good catalytic turnover and good conversion of the starting materials into the amino acid analogue.

Where the reduction is achieved by hydrogenation of the unsaturated dicarba bridge, the hydrogenation reaction may be performed in any solvent which provides good conversion of the starting materials into the desired amino acid analogue or peptide.

The solvent may be any polar solvent which does not adversely affect the hydrogenation catalyst or the yield of the amino acid analogue or peptide. Preferably, the solvent is an alcohol, such as methanol.

The reduction may be performed at any temperature ranging from reflux to room temperature. Preferably the metathesis reaction is conducted at ambient or room temperature.

Where the reduction is achieved by hydrogenation, the reduction is performed under hydrogen.

Other additives may also be added to the metathesis reaction.

Reaction Conditions for Tandem Metathesis-Reduction

The experimental conditions previously used for the reduction step of the tandem metathesis reduction require the use of high pressure (up to 30 bar, which is equivalent to about 440 Psi), high temperatures (such as reflux and 150° C.), and/or in the presence of a base (such as sodium hydride, lithium aluminium hydride, calcium hydride, sodium hydroxide, potassium carbonate, sodium hydroxide, potassium hydroxide, potash, potassium tert-butoxide or ammonia). However, it has been found that the catalyst residue following metathesis can perform the required hydrogenation reaction under mild hydrogen pressure, low temperature and without the addition of a base or an additional reagent to catalyse the reduction. The residue can also be left in air for several days and still generates an active hydrogenation catalyst on exposure to a hydrogen atmosphere.

The use of a base, particularly a strong base, is unsuitable for amino acid and peptide substrates. Accordingly, the present method involving tandem metathesis-reduction using a single catalyst under mild experimental conditions is essential for accessing chiral lipidic amino acids such as the amino acids of the present invention, and peptides containing them.

The reduction may be performed at a temperature below reflux or below 100° C. In some embodiments, the reduction may be performed at a temperature below 70° C., below 50° C., from below 50° C. to 0° C., from below 50° C. to room temperature, from 45° C. to 0° C., from 40° C. to room temperature, from 40° C. to 15° C. or preferably at room temperature or ambient temperature. Preferably the metathesis reaction is conducted at ambient or room temperature.

Where the reduction is achieved by hydrogenation, the reduction is performed under mild hydrogen pressure. Suitable hydrogen pressures include 100 Psi or less, below 100 Psi, below 90 Psi, below 80 Psi, below 70 Psi, from 100 to 40 Psi, or from 80 to 50 Psi.

The reduction may be performed in the absence of other additives. The reduction is performed in the absence of bases or strong bases including sodium hydride, lithium aluminium hydride, calcium hydride, sodium hydroxide, potassium carbonate, sodium hydroxide, potassium hydroxide, potash, potassium tert-butoxide or ammonia. The reduction may also be conducted without the need for addition of other catalysts, such as conventional reduction catalysts such at Pd/C, Pd(OAc)₂, PtO₂, and Wilkinson's catalyst.

Other additives may be added to the metathesis reaction.

The tandem metathesis-reduction may be performed in any solvent which provides good catalytic turnover and good conversion of the starting materials into the desired amino acid analogue or peptide.

Where the reduction is achieved by hydrogenation of the unsaturated dicarba bridge, the hydrogenation reaction may be performed in any solvent which provides good conversion of the starting materials into the desired amino acid analogue or peptide.

The solvent may be any polar solvent which does not adversely affect the metathesis and hydrogenation catalyst or the yield of the amino acid analogue or peptide. Preferably, the solvent is an alcohol, such as methanol.

Although column chromatography could be used to obtain final products with higher purity, the material obtained from the selective precipitation work-up is typically of sufficient purity to be directly used in solid phase peptide synthesis (SPPS).

Alternating Peptide Synthesis and Catalysis

In certain peptides it can be difficult to form intramolecular and intermolecular dicarba bridges due to deleterious aggregation and inappropriate positioning of the reactable (metathesisable) groups. The use of microwave radiation and/or the use of turn-inducing groups in the metathesis step (as discussed below) may facilitate the metathesis reaction and/or the reduction to occur, or occur more efficiently. However, for many peptide sequences, all of the existing strategies to enhance metathesis, even when used alone or in conjunction, can still fail to produce acceptable dicarba bridge yield.

In this approach, the sequence is grown in a stepwise fashion until both metathesisable residues have been incorporated. One of the peptides may be provided on a solid support. Preferably, the second metathesisable group of the pair is left at or near the N-terminus of the peptide. The resin-supported incomplete sequence is then exposed to the metathesis catalyst to form the dicarba bridge. Following the metathesis step, the resin can either be subjected to secondary catalysis (e.g. hydrogenation by tandem metathesis-hydrogenation), or followed immediately with the remaining SPPS to the N-terminus of the desired target peptide. It will be appreciated that this process can be conducted iteratively in order to introduce more than one dicarba bridge. This interrupted approach can be highly successful with sequences which are difficult to metathesise and/or reduce. The scheme below illustrates this approach.

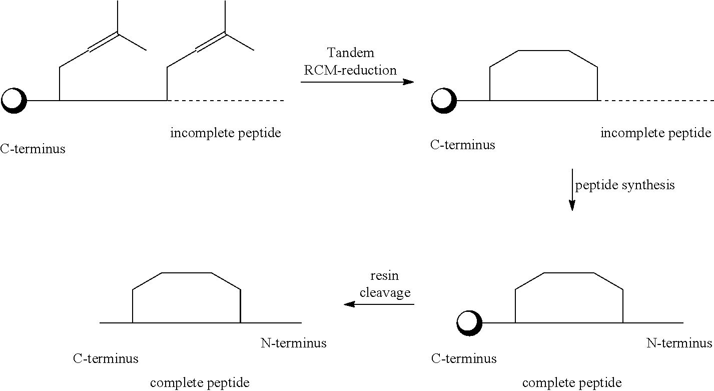

According to one embodiment, there is provided a method for the synthesis of a peptide or peptides containing a dicarba bridge, or a salt, solvate, derivative, isomer or tautomer thereof comprising the steps of:

(i) synthesising a reactable peptide containing a metathesisable group and a compound containing a complementary metathesisable group of formula (I′) or (II′), or a reactable peptide containing at least two metathesisable groups;
(ii) subjecting the reactable peptide and a compound formula (I′) or (II′), or the reactable peptides containing at least two metathesisable groups to metathesis in the presence of a reagent to catalyse the metathesis to form a dicarba bridge between the metathesisable groups;
(iii) reducing the dicarba bridge to form a saturated dicarba bridge, wherein the reagent used to catalyse step (i) also catalyses step (ii); and
(iii) adding one or more further amino acids to one or both ends of a the reactable peptide or the compound of formula (I′) or (II′).

Preferably, the peptide or peptides are synthesised to a point where the required metathesisable group is incorporated. The metathesisable group may be at or near one end of the reactable peptide or peptides. Preferably the metathesisable group is less than 5 residues and most suitably between 0-3 residues from one end of the reactable peptide. When the metathesisable group is 0 residues from one end of the reactable peptide, the metathesisable group is at the end of the reactable peptide.

In one example, where the peptide to be prepared is a dicarba analogue of a naturally occurring peptide or peptides, the method involves the synthesis of a part of the naturally occurring peptide. The part of the peptide or peptides that is synthesised in this step is the part or parts that contain at least two metathesisable groups. The peptide or peptides are then subjected to metathesis to form an unsaturated dicarba bridge. The peptide which is now joined by an unsaturated dicarba bridge is subjected to further peptide synthesis to produce the remainder of the desired peptide or to produce the target truncated peptide or peptides. The one or more further amino acids may be added to one or both ends of the reactable peptide. Where the method involves more than one reactable peptide, and the catalysis forms an intermolecular dicarba bridge, the one or more further amino acids may be added to one or both ends of any of the reactable peptides (e.g. added to one or both ends of either of the peptide chains connected by the intermolecular dicarba bridge).

Preferably, at least one of the reactable peptides is attached to a solid support.

In an embodiment of the present invention, at least one unsaturated dicarba bridge may be reduced to form an alkene-containing dicarba bridge or a saturated dicarba bridge. The step of reducing the dicarba bridge may occur before or after the step of adding one or more further amino acids to one or both ends of the at least one reactable peptide.

When a reactable peptide having an intramolecular bridge is desired, at least two complementary metathesisable groups are provided on a single peptide. Metathesis is conducted to form the dicarba bridge and then one or more further amino acids is added to one or both ends of at least one reactable peptide. Preferably, tandem metathesis-reduction is used to reduce the dicarba bridge before the addition of one or more further amino acids to one or both ends of the reactable peptide.

When a reactable peptide having an intermolecular bridge is desired, at least two reactable peptides have at least two complementary metathesisable groups between them (e.g. a reactable peptide containing a metathesisable group and a compound containing a complementary metathesisable group of formula (I′) or (II′), wherein the group X′ is a peptide). Metathesis is conducted to form the dicarba bridge between at least two complementary metathesisable groups forming a bridge between two reactable peptides, and then one or more further amino acids are added to one or both ends of at least one of the reactable peptides. Preferably, tandem metathesis-reduction is used to reduce the dicarba bridge before the addition of one or more further amino acids to one or both ends of at least one of the reactable peptides.

Microwave Reaction Conditions

It is possible to perform the metathesis reaction under microwave reaction conditions. This may assist the metathesis in addition to the advantages provided by the method. For instance, when the metathesisable groups are unblocked, but the arrangement, length or spatial orientation of the reactable organic compound prevents the metathesisable groups from being close enough to one another to enable the reaction to proceed. An alternative strategy is described below (see the description of “turn-inducing groups” below).

The microwave reaction conditions involve applying microwave radiation to the reactants (e.g. the amino acid or reactable peptide containing a metathesisable group and the compound of formula (I′) or (II′), containing a metathesisable group) in the presence of the metathesis catalyst for at least part of the reaction, usually for the duration of the reaction. Preferably, the reactable peptide or the compound of formula (I′) or (II′) is attached to a solid support. The microwave or microwave reactor may be of any type known in the art, operated at any suitable frequency. Typical frequencies in commercially available microwave reactors are 2.45 GHz, at a power of up to 500 W, usually of up to 300 W. The temperature of the reaction is preferably at elevated temperature, as a consequence of the microwave radiation, preferably at reflux, or around 100° C. The reaction is preferably performed in a period of not more than 5 hours, suitably for up to about 2 hours.

Turn-Inducing Groups

Another strategy which may further improve the performance of a metathesis reaction (in particular, ring closing metathesis) between two complementary metathesisable groups is the use of turn-inducing groups. This strategy is particularly useful for ring-closing metathesis where the metathesisable groups are located within a single peptide. As described above, this strategy can also be used in combination with microwave irradiation.

According to this embodiment, a reactable peptide is synthesised to contain a pair of unblocked complementary metathesisable groups, and a turn-inducing group located between the pair of complementary metathesisable groups. The turn-inducing group bends the backbone of the peptide for metathesis to form a dicarba bridge. Following metathesis or tandem metathesis-reduction, one or more further amino acids are added to either end of the reactable peptide.

The peptide backbone in α-peptides is generally linear as the component amino acids (especially when these are the 20 common amino acids, with exception of proline) form trans-configuration peptide bonds. Proline, a pyrrolidine analogue, can induce a turn in an otherwise linear peptide. This is a naturally-occurring turn-inducing group. This embodiment is particularly suited to those peptides that do not contain a naturally-occurring turn-inducing amino acid, such as proline.

Preferably the turn-inducing group is a turn-inducing amino acid, dipeptide or protein, and is preferably synthetic (non-naturally occurring). Examples of suitable synthetic turn-inducing amino acids are the pseudoprolines, including derivatives of serine, threonine and cysteine (shown below). The pseudoprolines have been derivatised to contain a cyclic group between the amino acid sidechain (via the —OH or —SH group), and the amino nitrogen atom. A typical derivatising agent is CH3-C(═O)—CH3, such that the turn-inducing amino acids are:

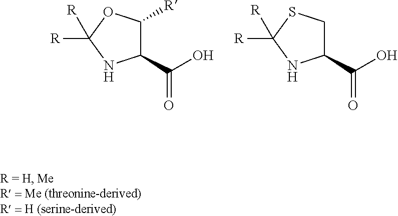

These turn-inducing residues are often prepared as dipeptide units to aid incorporation into peptides. An example of a suitable turn-inducing residue is 5,5-dimethylproline which is stable and may stay in the peptide permanently. However, after metathesis, some pseudoproline(s) may be converted back to the underivatised amino acid (serine, threonine or cysteine) by removal of the derivatising agent usually on treatment with acid. The conditions for cleavage of the peptide from a solid support will usually achieve this.

If, for example, the turn-inducing amino acid is one of pseudo-serine, pseudo-proline or pseudo-cysteine, then the method may further comprise the step of converting the pseudo-serine, pseudo-proline or pseudo-cysteine to serine, proline or cysteine, respectively.

The use of pseudoproline residues can be combined with the other preferred features described herein. As one example, pseudo-proline residues can be used in combination with microwave conditions.

As described above, a turn inducing residue is provided between the two complementary metathesisable groups of the reactable peptide which will form the dicarba bridge, in order to bring them closer together during the metathesis step.

Tethers Between Peptide Sequences

In some instances, cross-metathesis between peptide sequences can be difficult and low yielding. Success is often sequence dependent and relies on favourable positioning of reacting motifs which can be hampered by peptide size, aggregation, deleterious hydrogen bonding/salt bridges and steric constraints imposed by the primary sequence.

One approach by which we can enhance the metathesis between two complementary metathesisable groups is to utilise a contiguous peptide sequence, containing the two amino acids or peptides to be connected by a dicarba bridge, joined together via a removable tether. Such an approach capitalises on the improved positioning of the reactive motifs imposed by the tether and hence exploits the enhanced reactivity via an intramolecular reaction (RCM) compared to an intermolecular reaction (CM) to produce superior ligation yields. Such an approach is illustrated below:

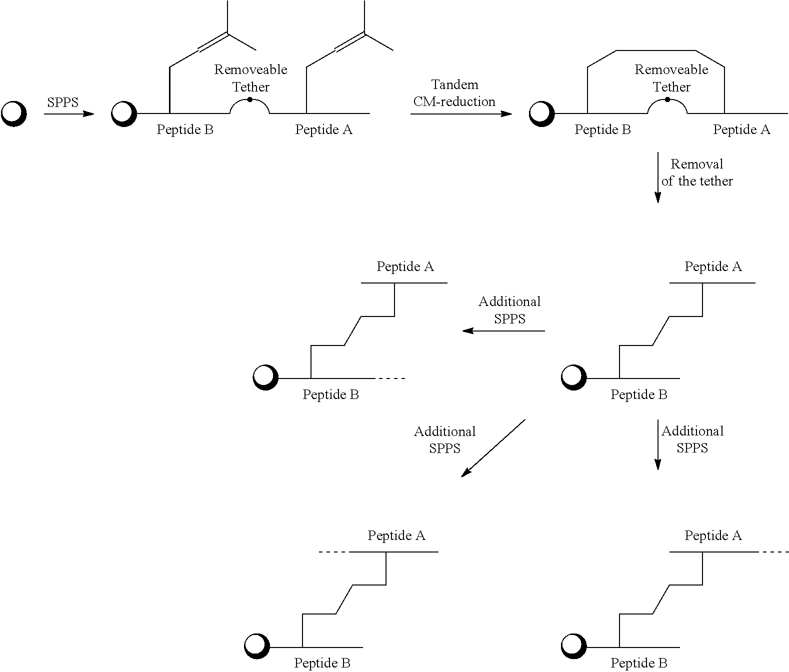

In this example, SPPS is used to generate a single peptide sequence where a transient/removable tether is positioned between the two metathesisable groups. Catalysis is then performed on the resin-bound peptide (RCM, RCAM and/or H or tandem metathesis-reduction) and the resultant cyclic peptide is then cleaved open at the tether to result in the target acyclic peptide. The final peptide is analogous to that produced via a direct CM reaction between two peptide sequences. The resin-appended sequence can then be further elaborated via SPPS in a number of positions as shown above.

Groups which may function as a removable tether are structurally diverse. The removable tether may be any motif which can be chemoselectively incorporated and removed from the sequence, either chemically or enzymatically. The removable tether may be a motif which can be added by reductive amination. The removable tether may be a motif which can be removed by photolysis. Preferably, the removable tether is a motif which also promotes a turn in the backbone of the primary sequence (similarly for the turn-inducing residues described above). In this approach, the metathesis reaction may be enhanced by suitable positioning of the reactive motifs. As one example, the removable tether may be hydroxy-6-nitrobenzaldehyde.

Amino Acid Analogues

Some of the amino acid analogues prepared by the method of the present invention are new.

In one embodiment there is provided an amino acid analogue of the formula (VIII):

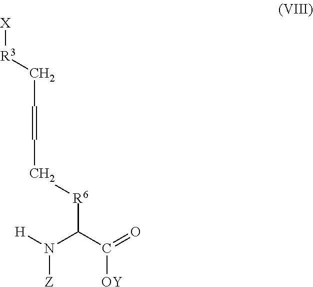

The group R³ is either present or absent. When R³ is present it is a divalent linker between the metathesisable group and the group X. When R³ is absent, the divalent methylene group adjacent the alkene double bond in formula (VIII) is the linker between the metathesisable group and the group X.

The group R⁶ is either present or absent. When R⁶ is present it is a divalent linker between the metathesisable group and the amino acid backbone. When R⁶ is absent, the divalent methylene group adjacent the alkene double bond in formula (VIII) or the divalent methylene group adjacent the alkyne double bond in formula (IX) is the linker between the metathesisable group and the amino acid backbone.

In one embodiment, at least one of R³ and R⁶ is present. In another embodiment, both R³ and R⁶ are present.

When R³ or R⁶ are present, they are independently selected from a heteroatom, a substituted or unsubstituted C₁ to C₂₀ alkyl, and a substituted or unsubstituted C₁ to C₂₀ alkyl group interrupted by one or more heteroatoms. Preferably, both R³ and R⁶ are present. More preferably, R³ and R⁶ are not both unsubstituted alkyl.

In one embodiment, R³ and R⁶ are not both unsubstituted alkyl.

When the group R³ or R⁶ is a heteroatom, the heteroatom is preferably oxygen, sulfur, nitrogen or phosphorus. When the heteroatom is a nitrogen, it is preferably protected, provided as a quaternary amine salt or avoided during methathesis. The heteroatom may be selected from the group consisting of O, S(O), S(O)₂, SO₂NH, OS(O₂)O, NH, N(R⁷), PO₄, and P(R⁷)₂, wherein each R⁷ is independently a substituted or unsubstituted C₁ to C₁₀ alkyl.

When the group R³ or R⁶ is a substituted or unsubstituted alkyl, it is preferably an alkyl group having from 1 to about 20 carbon atoms, from 1 to 15 carbon atoms, from 1 to 8 carbon atoms, from 1 to 6 carbon atoms or from 1 to 4 carbon atoms provided that both R³ and R⁶ are not unsubstituted alkyl. More preferably, R³ or R⁶ is an unsubstituted alkyl group having from 1 to 8 carbon atoms.

When the group R³ or R⁶ is a substituted or unsubstituted alkyl interrupted by one or more heteroatoms, preferably, the alkyl group has from 1 to about 20 carbon atoms, from 1 to 15 carbon atoms, from 1 to 8 carbon atoms, from 1 to 6 carbon atoms or from 1 to 4 carbon atoms and is interrupted by from 1 to about 20 heteroatoms, from 1 to 15 heteroatoms atoms, from 1 to 8 heteroatoms, from 1 to 6 heteroatoms or from 1 to 4 heteroatoms. The heteroatoms may be selected from the group consisting of N, O, S, P and mixtures thereof. When the heteroatom is a nitrogen, it is preferably protected, provided as a quaternary amine salt or avoided during methathesis. More preferably, R³ and/or the group R⁶ is an alkyl group having from 1 to 8 carbon atoms that is interrupted by from 1 to 3 heteroatoms.

When R³ and/or the group R⁶ is a substituted alkyl or substituted alkyl interrupted by one or more heteroatoms, the substituents are groups that do not poison the metathesis catalyst or affect its selectivity. Preferably, the substituents may be selected from esters, carbonyls (oxo) including aldehydes and ketones, carboxyls, amides, nitriles and alcohols.

The group Z is selected from H, a salt and a protecting group. When Z is a protecting group, it may be selected from the group consisting of 9-fluorenylmethyl carbamate (Fmoc), 2,2,2-trichloroethyl carbamate (Troc), t-butyl carbamate (Boc), allyl carbamate (Alloc), 2-trimethylsilylethyl (Teoc) and benzyl carbamate (Cbz). Preferably the group Z is Fmoc.

The group Y is selected from H and a protecting group. When Y is a protecting group, it may be an ester such as an alkyl ester, for example, methyl ester, ethyl ester, t-Bu ester or a benzyl ester.

X is independently an H or an effector molecule as described above.

In another embodiment there is provided an amino acid analogue of the formula (X):

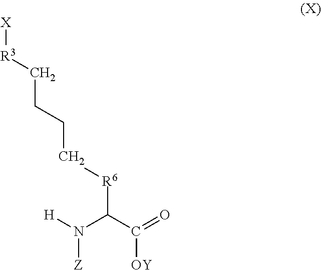

The group R³ is either present or absent. When R³ is present it is a divalent linker between the metathesisable group and the group X. When R³ is absent, the divalent methylene group adjacent the alkene double bond in formula (VIII) or the divalent methylene group adjacent the corresponding alkyl bond in formula (X) is the linker between the metathesisable group and the group X. If R³ is absent, it is preferable that the group R⁶ is present.

The group R⁶ is either present or absent. When R⁶ is present it is a divalent linker between the metathesisable group and the amino acid backbone. When R⁶ is absent, the divalent methylene group adjacent the alkene double bond in formula (VIII) or the divalent methylene group adjacent the corresponding alkyl bond in formula (X) is the linker between the metathesisable group and the amino acid backbone. If R⁶ is absent, it is preferable that the group R³ is present.

In one embodiment, at least one of R³ and R⁶ is present. In another embodiment, both R³ and R⁶ are present.

When R³ and/or R⁶ are present, they are independently selected from a heteroatom, a substituted or unsubstituted C₁ to C₂₀ alkyl, and a substituted or unsubstituted C₁ to C₂₀ alkyl group interrupted by one or more heteroatoms.

In one embodiment, R³ and R⁶ are not both unsubstituted alkyl.

When the group R³ or R⁶ is a heteroatom, the heteroatom is preferably oxygen, sulfur, nitrogen or phosphorus. When the heteroatom is a nitrogen, it is preferably protected, provided as a quaternary amine salt or avoided during methathesis. The hetero atom may be selected from the group consisting of O, S(O), S(O)₂, SO₂NH, OS(O₂)O, NH, N(R⁷), PO₄, and P(R⁷)₂, wherein each R⁷ is independently a substituted or unsubstituted C₁ to C₁₀ alkyl.

When the group R³ or R⁶ is a substituted or unsubstituted alkyl, it is preferably an alkyl group having from 1 to about 20 carbon atoms, from 1 to 15 carbon atoms, from 1 to 8 carbon atoms, from 1 to 6 carbon atoms or from 1 to 4 carbon atoms. Preferably, R³ or R⁶ is an unsubstituted alkyl group having from 1 to 8 carbon atoms.

When the group R³ or R⁶ is a substituted or unsubstituted alkyl interrupted by one or more heteroatoms, preferably, the alkyl group has from 1 to about 20 carbon atoms, from 1 to 15 carbon atoms, from 1 to 8 carbon atoms, from 1 to 6 carbon atoms or from 1 to 4 carbon atoms and is interrupted by from 1 to about 20 heteroatoms, from 1 to 15 heteroatoms atoms, from 1 to 8 heteroatoms, from 1 to 6 heteroatoms or from 1 to 4 heteroatoms. The heteroatoms may be selected from the group consisting of N, O, S, P and mixtures thereof. When the heteroatom is a nitrogen, it is preferably protected, provided as a quaternary amine salt or avoided during methathesis. More preferably, R³ and/or the group R⁶ is an alkyl group having from 1 to 8 carbon atoms that is interrupted by from 1 to 3 heteroatoms.

When R³ or R⁶ is a substituted alkyl or substituted alkyl interrupted by one or more heteroatoms, the substituents are groups that do not poison the metathesis catalyst or affect its selectivity. Preferably, the substituents may be selected from esters, carbonyls (oxo) including aldehydes and ketones, carboxyls, amides, nitriles and alcohols.

The group Z is selected from H, a salt and a protecting group. When Z is a protecting group, it may be selected from the group consisting of 9-fluorenylmethyl carbamate (Fmoc), 2,2,2-trichloroethyl carbamate (Troc), t-butyl carbamate (Boc), allyl carbamate (Alloc), 2-trimethylsilylethyl (Teoc) and benzyl carbamate (Cbz). Preferably the group Z is Fmoc.

The group Y is selected from H and a protecting group. When Y is a protecting group, it may be an ester such as an alkyl ester, for example, methyl ester, ethyl ester, t-Bu ester or a benzyl ester.

X is independently an H or an effector molecule as described above.

Peptides

Proteins and peptides are oligomers of amino acids that mediate a diverse array of functions within living systems. They exist as hormones, biochemical inhibitors, antigens, growth factors and transmembrane carriers. The high biological activity of many peptides makes them particularly attractive pharmaceutical targets. However, the clinical development of orally active peptide drugs continues to be restricted by their unfavorable physicochemical properties. Their poor resistance to proteolytic enzymes, rapid excretion through the liver and kidneys, their inability to cross membrane barriers, such as the intestinal and blood-brain barriers, and in some cases, their low solubility and tendency to aggregate, have contributed to the poor bioavailability of peptide-based therapeutics. Successful therapeutic application of peptides, therefore requires the design and synthesis of novel peptidomimetics which possess improved physicochemical properties and uncompromised biological activity.

One class of peptides that are of particular interest are the peptidomimetics—that is, a peptide that has a series of amino acids that mimics identically or closely a naturally occurring peptide, but with at least amino acid of formula (VIII) or (X), and optionally one or more further differences, such as the removal of a cystine bridge, the presence of one or more dicarba bridges or a change by up to 20% of the amino acids in the sequence, as non-limiting examples. The amino acid analogue may, for example, replace one or more of the naturally occurring amino acids in the native peptide sequence, or may, for example, replace one or more naturally occurring disulfide bridges or replace one or more non-covalent interactions present in the peptide, such as salt bridges or non-covalent interactions involved in secondary structure motifs such as α-helices or β-sheets. In identically or closely mimicking a naturally occurring peptide, the peptidomimetics having at least one dicarba bridge may optionally include one or more differences from the natural peptide, such as the removal of a cystine bridge, a change by up to 20% of the amino acids in the sequence, the inclusion of non-natural amino acids, D-amino acids or β-amino acids as non-limiting examples. Of particular interest are dicarba analogues of naturally-occurring disulfide-containing peptides, in which one or more of the disulfide bonds are replaced with dicarba bridges. These may also be classed as pseudo-peptides.

The method of the present invention can be used to prepare new amino acid analogues which can be incorporated into a sequence and can be designed to provide the peptide with improved properties, such as improved physicochemical properties, improved bioavailability, improved membrane permeability or improved solubility. Preferably, modulation of these properties can be achieved by altering the nature of the groups R³, R⁶ and X. For example, including an amino acid of the present invention in which the group R⁶ is an extended alkyl chain (for example, C₄ to C₈) could improve hydrophobicity of a peptide, while inclusion of an amino acid of the present invention in which the group R⁶ is an alkyl chain interrupted with one or more heteroatoms (for example, a polyethylene glycol chain including from 4 to 8 carbons and 1 to 3 oxygen atoms) could improve hydrophilicity of the peptide. As another example, an amino acid of the present invention in which the group X is a therapeutic agent or prodrug could improve hydrophobicity of the therapeutic agent or drug when the amino acid analogue is included in a peptide.

One advantage associated with the amino acid analogues described above is that they may be incorporated into a peptide sequence without further purification. Furthermore, the amino acid analogues may be produced by the method of the present invention in sufficient purity to be directly used in solid phase peptide synthesis (SPPS). The method of the present invention also provides more accessible chiral, non-proteinaceous amino acids, which can be used to provide a greater variety of peptidomimetics.

In one embodiment, the present invention provides a peptide, which contains an amino acid residue of formula (VIII¹):

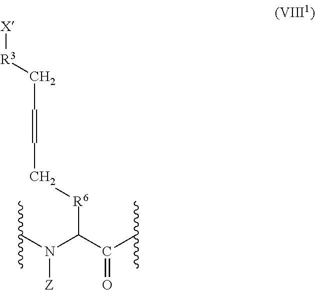

at any position in the peptide sequence.

The group R³ is either present or absent. When R³ is present it is a divalent linker between the metathesisable group and the group X′. When R³ is absent, the divalent methylene group adjacent the alkene double bond in formula (VIII¹) is the linker between the metathesisable group and the group X′.

The group R⁶ is either present or absent. When R⁶ is present it is a divalent linker between the metathesisable group and the amino acid backbone. When R⁶ is absent, the divalent methylene group adjacent the alkene double bond in formula (VIII¹) is the linker between the metathesisable group and the amino acid backbone.

In one embodiment, at least one of R³ and R⁶ is present. In another embodiment, both R³ and R⁶ are present.

When R³ or R⁶ are present, they are independently selected from a heteroatom, a substituted or unsubstituted C₁ to C₂₀ alkyl, and a substituted or unsubstituted C₁ to C₂₀ alkyl group interrupted by one or more heteroatoms. Preferably, both R³ and R⁶ are present. More preferably, R³ and R⁶ are not both unsubstituted alkyl.

In one embodiment, R³ and R⁶ are not both unsubstituted alkyl.

When the group R³ or R⁶ is a heteroatom, the heteroatom is preferably oxygen, sulfur, nitrogen or phosphorus. When the heteroatom is a nitrogen, it is preferably protected, provided as a quaternary amine salt or avoided during methathesis. The hetero atom may be selected from the group consisting of O, S(O), S(O)₂, SO₂NH, OS(O₂)O, NH, N(R⁷), PO₄, and P(R⁷)₂, wherein each R⁷ is independently a substituted or unsubstituted C₁ to C₁₀ alkyl.

When the group R³ or R⁶ is a substituted or unsubstituted alkyl, it is preferably an alkyl group having from 1 to about 20 carbon atoms, from 1 to 15 carbon atoms, from 1 to 8 carbon atoms, from 1 to 6 carbon atoms or from 1 to 4 carbon atoms provided that both R³ and R⁶ are not unsubstituted alkyl. More preferably, R³ or R⁶ is an unsubstituted alkyl group having from 1 to 8 carbon atoms.

When the group R³ or R⁶ is a substituted or unsubstituted alkyl interrupted by one or more heteroatoms, preferably, the alkyl group has from 1 to about 20 carbon atoms, from 1 to 15 carbon atoms, from 1 to 8 carbon atoms, from 1 to 6 carbon atoms or from 1 to 4 carbon atoms and is interrupted by from 1 to about 20 heteroatoms, from 1 to 15 heteroatoms atoms, from 1 to 8 heteroatoms, from 1 to 6 heteroatoms or from 1 to 4 heteroatoms. The heteroatoms may be selected from the group consisting of N, O, S, P and mixtures thereof. When the heteroatom is a nitrogen, it is preferably protected, provided as a quaternary amine salt or avoided during methathesis. More preferably, R³ and/or the group R⁶ is an alkyl group having from 1 to 8 carbon atoms that is interrupted by from 1 to 3 heteroatoms.

When R³ and/or the group R⁶ is a substituted alkyl or substituted alkyl interrupted by one or more heteroatoms, the substituents are groups that do not poison the metathesis catalyst or affect its selectivity. Preferably, the substituents may be selected from esters, carbonyls (oxo) including aldehydes and ketones, carboxyls, amides, nitriles and alcohols.

The group Z is selected from H, a salt and a protecting group. When Z is a protecting group, it may be selected from the group consisting of 9-fluorenylmethyl carbamate (Fmoc), 2,2,2-trichloroethyl carbamate (Troc), t-butyl carbamate (Boc), allyl carbamate (Alloc), 2-trimethylsilylethyl (Teoc) and benzyl carbamate (Cbz). Preferably the group Z is Fmoc.

X′ is independently H, an effector molecule, an amino acid or a peptide. When X′ is an amino acid or a peptide, there is a dicarba bridge between the amino acid or peptide containing a metathesisable group and a compound of formula (I), (II) or (III). For example, the dicarba bridge may be formed between two separate peptide chains (the reactable peptide and the compound in which X′ is a peptide) to form an interchain dicarba bridge. This strategy can be used to replace naturally occurring disulfide bridges present between peptide subunits with dicarba bridges.

The method of the present invention can also be used to prepare new peptides containing a dicarba bridge either between two peptides or within a single peptide. In one embodiment, the method uses a reactable peptide containing a metathesisable group and a compound containing a complementary metathesisable group of formula (I′) or (II′) to form a dicarba bridge between the reactable peptide and the compound of formula (I′) or (II′). In another embodiment, the method uses a reactable peptide containing at least two metathesisable groups to form a dicarba bridge within the reactable peptide.

In one embodiment there is provided a peptide or peptides containing a dicarba bridge or a salt, solvate, derivative, isomer or tautomer thereof containing an amino acid residue of the formula (X¹):

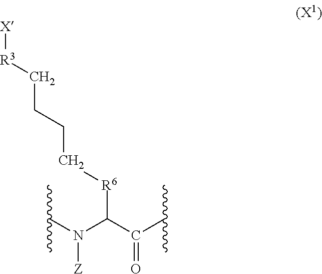

The group R³ is either present or absent. When R³ is present it is a divalent linker between the metathesisable group and the group X′. When R³ is absent, the divalent methylene group adjacent the alkene double bond in formula (VIII¹) or the divalent methylene group adjacent the corresponding alkyl bond in formula (X¹) is the linker between the metathesisable group and the group X. If R³ is absent, it is preferable that the group R⁶ is present.

The group R⁶ is either present or absent. When R⁶ is present it is a divalent linker between the metathesisable group and the amino acid backbone. When R⁶ is absent, the divalent methylene group adjacent the alkene double bond in formula (VIII¹) or the divalent methylene group adjacent the corresponding alkyl bond in formula (X¹) is the linker between the metathesisable group and the amino acid backbone. If R⁶ is absent, it is preferable that the group R³ is present.

In one embodiment, at least one of R³ and R⁶ is present. In another embodiment, both R³ and R⁶ are present.

When R³ or R⁶ are present, they are independently selected from a heteroatom, a substituted or unsubstituted C₁ to C₂₀ alkyl, and a substituted or unsubstituted C₁ to C₂₀ alkyl group interrupted by one or more heteroatoms. Preferably, both R³ and R⁶ are present. More preferably, R³ and R⁶ are not both unsubstituted alkyl.

In one embodiment, R³ and R⁶ are not both unsubstituted alkyl.

When the group R³ or R⁶ is a heteroatom, the heteroatom is preferably oxygen, sulfur, nitrogen or phosphorus. When the heteroatom is a nitrogen, it is preferably protected, provided as a quaternary amine salt or avoided during methathesis. The heteroatom may be selected from the group consisting of O, S(O), S(O)₂, SO₂NH, OS(O₂)O, NH, N(R⁷), PO₄, and P(R⁷)₂, wherein each R⁷ is independently a substituted or unsubstituted C₁ to C₁₀ alkyl.

When the group R³ or R⁶ is a substituted or unsubstituted alkyl, it is preferably an alkyl group having from 1 to about 20 carbon atoms, from 1 to 15 carbon atoms, from 1 to 8 carbon atoms, from 1 to 6 carbon atoms or from 1 to 4 carbon atoms provided that both R³ and R⁶ are not unsubstituted alkyl. More preferably, R³ or R⁶ is an unsubstituted alkyl group having from 1 to 8 carbon atoms.

When the group R³ or R⁶ is a substituted or unsubstituted alkyl interrupted by one or more heteroatoms, preferably, the alkyl group has from 1 to about 20 carbon atoms, from 1 to 15 carbon atoms, from 1 to 8 carbon atoms, from 1 to 6 carbon atoms or from 1 to 4 carbon atoms and is interrupted by from 1 to about 20 heteroatoms, from 1 to 15 heteroatoms atoms, from 1 to 8 heteroatoms, from 1 to 6 heteroatoms or from 1 to 4 heteroatoms. The heteroatoms may be selected from the group consisting of N, O, S, P and mixtures thereof. When the heteroatom is a nitrogen, it is preferably protected, provided as a quaternary amine salt or avoided during methathesis. More preferably, R³ and/or the group R⁶ is an alkyl group having from 1 to 8 carbon atoms that is interrupted by from 1 to 3 heteroatoms.

When R³ and/or the group R⁶ is a substituted alkyl or substituted alkyl interrupted by one or more heteroatoms, the substituents are groups that do not poison the metathesis catalyst or affect its selectivity. Preferably, the substituents may be selected from esters, carbonyls (oxo) including aldehydes and ketones, carboxyls, amides, nitriles and alcohols.

The group Z is selected from H, a salt and a protecting group. When Z is a protecting group, it may be selected from the group consisting of 9-fluorenylmethyl carbamate (Fmoc), 2,2,2-trichloroethyl carbamate (Troc), t-butyl carbamate (Boc), allyl carbamate (Alloc), 2-trimethylsilylethyl (Teoc) and benzyl carbamate (Cbz). Preferably the group Z is Fmoc.

X′ is independently H, an effector molecule, an amino acid or a peptide. When X′ is an amino acid or a peptide, there is a dicarba bridge between the amino acid or peptide containing a metathesisable group and a compound of formula (I), (II) or (III). For example, the dicarba bridge may be formed between two separate peptide chains (the reactable peptide and the compound in which X′ is a peptide) to form an interchain dicarba bridge. This strategy can be used to replace naturally occurring disulfide bridges present between peptide subunits with dicarba bridges.

Peptide Synthesis

Peptides are synthesized by coupling the carboxyl group or C-terminus of one amino acid to the amino group or N-terminus of another. Due to the possibility of unintended reactions, protecting groups are usually necessary.

Peptide synthesis using the amino acid analogues of the present invention can be performed using any method known to a person skilled in the art, such as, for example, using solid phase peptide synthesis (SPPS) or solution phase peptide synthesis. Automated peptide synthesizers may be used or the synthesis may be performed manually.

SPPS involves repeated cycles of coupling-wash-deprotection-wash. The free N-terminal amine of a solid-phase attached peptide is coupled to a single N-protected amino acid unit. This unit is then deprotected, revealing a new N-terminal amine to which a further amino acid may be attached.

At any point in the process, the amino acid used may be an amino acid analogue of formula (VIII), (IX) or (X) as defined above.

In one embodiment, there is provided a method for preparing a peptide containing an amino acid analogue of formula (VIII), (IX) or (X), which comprises synthesising a peptide by stepwise addition of amino acid residues to produce the peptide, wherein one or more of the amino acid residues is an amino acid analogue of formula (VIII), (IX) or (X).

In another embodiment, there is provided a method for preparing a peptide containing an amino acid residue of formula (X¹) or a salt, solvate, derivative, isomer or tautomer thereof,

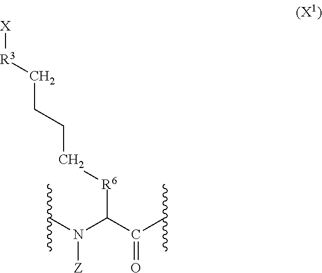

comprising the steps of:
(i) subjecting an amino acid containing a metathesisable group of formula (VI) to metathesis with a compound containing a complementary metathesisable group of formula (I) or (II):

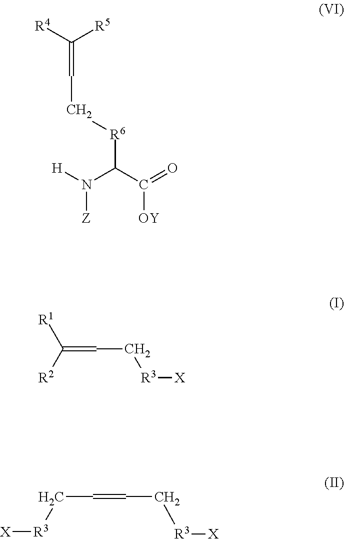

wherein R¹, R², R⁴ and R⁵ are independently selected from H and substituted or unsubstituted C₁ to C₄ alkyl; each R³ is either absent or independently selected from a heteroatom, a substituted or unsubstituted C₁ to C₂₀ alkyl, and a substituted or unsubstituted C₁ to C₂₀ alkyl group interrupted by one or more heteroatoms; R⁶ is either absent or selected from a heteroatom, a substituted or unsubstituted C₁ to C₂₀ alkyl, and a substituted or unsubstituted C₁ to C₂₀ alkyl interrupted by one or more heteroatoms; Z is selected from H, a salt and a protecting group; Y is selected from H and a protecting group; and each X is independently selected from H and an effector molecule; in the presence of a reagent to catalyse the metathesis to form a dicarba bridge between the amino acid of formula (VI) and the compound of formula (I) or (II);
(ii) reducing the dicarba bridge to form a saturated dicarba bridge, wherein the reagent used to catalyse step (i) also catalyses step (ii); and
(iii) synthesising a peptide by stepwise addition of amino acid residues to produce the peptide, wherein one or more of the amino acid residues is of formula (X¹).

The present invention also provides the peptide or a salt, solvate, derivative, isomer or tautomer thereof synthesised by the methods as described above.

Although the remainder of the description refers to particular examples or embodiments of the invention, it is to be understood that modifications or improvements may be made thereto without departing from the scope of the invention.

### EXAMPLES

The invention is described further by reference to the following non-limiting examples of the invention.

Method of Preparing Amino Acid Analogues

Methodology for the generation of amino acid analogues has been developed, examples of which are shown in Scheme 1. The resultant amino acid analogues can be directly incorporated into peptide sequences.

This methodology can be used to alter the properties of an amino acid (e.g. by increasing the lipidity or hydrophilicity of an amino acid), to incorporate effector molecules such as, for example, the incorporation of cholesterol (in Scheme 1, line 2) and nitrogen-bearing chelates for radio labelling of peptides (in Scheme 1, line 3).

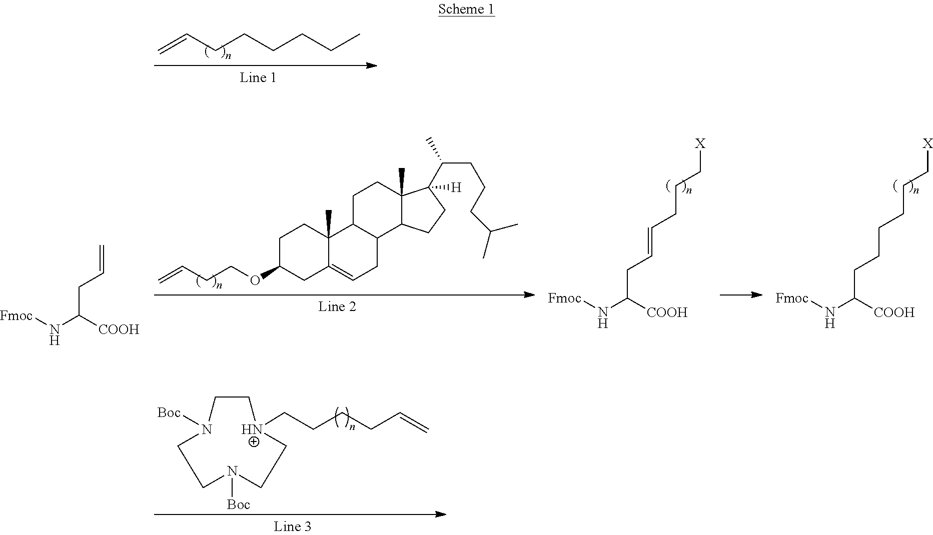

One example of the present method involves Ru-alkylidene catalysed tandem cross metathesis (CM)/hydrogenation route to lipoamino acids (Scheme 2). This process utilises commercially available N-Fmoc- and N-Boc-derivatives of allylglycine, 3a and 3b respectively, and the resultant amino acid analogues can be directly incorporated into peptide sequences without chromatography. The carboxylic acid functionality in 3 is also well tolerated by Ru-based metathesis catalysts which eliminates unnecessary functional group protection and deprotection steps from the reaction process. It has also been found that the stereogenic centre adjacent to the carbonyl functionality is not epimerised under mild Ru-alkylidene catalysed metathesis reactions.

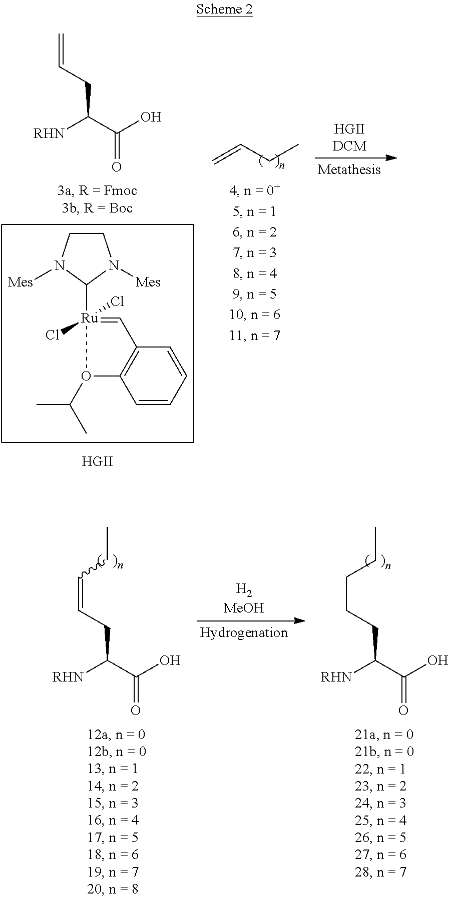

Cross metathesis of commercially available N-protected-allylglycine 3a and 3b with terminal alkenes 4-11 was extremely sensitive towards reaction conditions. Optimisation of the CM reaction was performed using N-Fmoc-protected allylglycine 3a and 1-decene 11. Reactions performed in DCM or EtOAc heated at reflux yielded not only the desired cross product 19 but also sidechain extended and truncated homologues 18 and 20 (Table 1). Formation of these by-products suggested that concomitant olefin isomerisation and secondary metathesis processes were occurring marring an otherwise efficient process. The formation of the undesired homologues may be eliminated to prevent the need for downstream purification. Towards this end, various reaction conditions, solvents and additives were screened to minimize side reactions.

Reactions performed at ambient temperature yielded only the target cross product 19 (Entry 3). Altering the stoichiometry, addition of molecular sieves and performing the reaction in EtOAc, while maintaining the reaction at room temperature, did not affect conversion (Entries 4-9). When the reaction was performed with a continuous flow of nitrogen through the head space, an increase in conversion was observed (Entry 10). While not wishing to be bound by theory, it appears that the continuous nitrogen flow facilitated efficient removal of ethylene, an expected by-product generated during the CM, and concentrated the reagents during the course of the reaction to drive the equilibrium towards the target product 19.

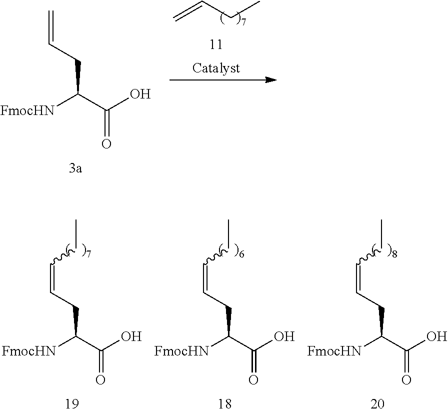

TABLE 1
Optimisation of the CM reaction

| | | Equiva- | | | Conversion | By- |
|---|---|---|---|---|---|---|
| | | lence of | | Con- | to 19 | products |
| Entry | Catalyst | 11 | Solvent | ditions | (%) | 18 + 20 |
| 1 | HGII | 5 | DCM | Δ | 93 | Yes |
| 2 | HGII | 5 | EtOAc | Δ | 90 | Yes |
| 3 | HGII | 5 | DCM | r.t. | 66 | No |
| 4 | GII | 5 | DCM | r.t. | 52 | No |
| 5 | HGII | 5 | EtOAc | r.t. | 62 | Trace |
| 6 | HGII | 5 | DCM | r.t.^{a} | 52 | No |
| 7 | HGII | 1 | DCM | r.t. | 32 | No |
| 8 | HGII | 2 | DCM | r.t. | 65 | No |
| 9 | HGII | 10 | DCM | r.t. | 70 | No |
| 10 | HGII | 5 | DCM | r.t.^{b} | 87 | No |

^{a}Molecular sieves added to the reaction.
^{b}Reaction performed with a N₂ bleed.

With the optimised CM conditions in hand, synthesis of the N-protected-alkylglycine series (n=0→7) commenced. Cross metathesis of allylglycine derivatives 3a and 3b with 1-alkenes 4-11 gave the alkene intermediates 12-20 as a mixture of E- and Z-isomers. Metathesis conversion was monitored by ¹H n.m.r. spectroscopy and found to be complete over 16 hours. Without workup, the mixture was subjected to hydrogenation.

After completion of the CM reaction, MeOH was added to the residue material and the solutions transferred to a Fischer-Porter tube. The tandem Ru-catalysed hydrogenation was performed at 60 p.s.i. at ambient temperature and yielded the saturated amino acid analogues 21-28 in good overall yield (Table 2). Furthermore, the two step, tandem CM/hydrogenation process could also be easily scaled. Compound 26 was synthesised on gram scale with yields comparable to small scale reactions.

TABLE 2
Tandem CM/hydrogenation yields

| Entry | Product | Overall yield (%) |
|---|---|---|
| 1 | 21a | 93^{†} |
| 2 | 21b | 78 |
| 3 | 22 | 86 |
| 4 | 23 | 65 |
| 5 | 24 | 81 |
| 6 | 25 | 73 |
| 7 | 26 | 76 |
| 8 | 27 | 80 |
| 9 | 28 | 75 |

^{†}Non-terminal cis-2-butene was used as a 1-propene equivalent in this reaction

Isolation and purification of the saturated N-Fmoc-amino acids 21-28 was achieved via selective precipitation. Whilst column chromatography could be used to obtain final products with higher purity, the material obtained from the selective precipitation work-up is of sufficient purity to be directly used in solid phase peptide synthesis (SPPS). Despite the lipophilic character of the C9 side chain in 26, incorporation of the N-Fmoc-protected residue 26 into a pentapeptide sequence 29 was achieved. LC-MS analysis of the material obtained after cleavage from the resin showed one major peak in the chromatogram which corresponded in mass to peptide 29, the target sequence.

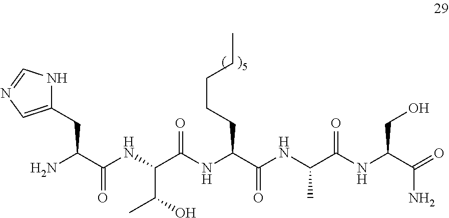

General Experimental

Instrumentation

Melting points (m.p.) were determined using a Reichert hot-stage melting point apparatus and are uncorrected.

Infrared spectra (IR) spectra were recorded on a Perkin-Elmer 1600 series Fourier Transform infrared spectrophotometer as thin films of liquid (neat) between sodium chloride plates. IR absorptions (vₘₐₓ) are reported in wavenumbers (cm⁻¹) with the relative intensities expressed as s (strong), m (medium), w (weak) or prefixed b (broad).

Proton nuclear magnetic resonance (¹H n.m.r.) spectra were recorded on a Bruker DRX400 spectrometer operating at 400 MHz, as solutions in deuterated solvents as specified. Each resonance was assigned according to the following convention: chemical shift (rotamers); multiplicity; number of protons; observed coupling constants (J Hz) and proton assignment. Chemical shifts (δ), measured in parts per million (ppm), are reported relative to the residual proton peak in the solvent used as specified. Multiplicities are denoted as singlet (s), doublet (d), triplet (t), quartet (q), pentet (p), multiplet (m) or prefixed broad (b), or a combination where necessary.

Carbon-13 nuclear magnetic resonance (¹³C n.m.r.) spectra were recorded on a Bruker DRX400 spectrometer operating at 100 MHz, as solutions in deuterated solvents as specified. Chemical shifts (δ), measured in parts per million (ppm), are reported relative to the residual proton peak in the deuterated solvent (as specified).

Low resolution electrospray ionisation (ESI) mass spectra were recorded on a Micromass Platform Electrospray mass spectrometer (QMS-quadrupole mass electrometry) as solutions in specified solvents. Spectra were recorded in positive and negative modes (ESI⁺ and ESI⁻) as specified. High resolution electrospray mass spectra (HRMS) were recorded on a Bruker BioApex 47e Fourier Transform mass spectrometer (4.7 Tesla magnet) fitted with an analytical electrospray source. The mass spectrometer was calibrated with an internal standard solution of sodium iodide in MeOH.

Analytical normal phase high performance liquid chromatography (NP-HPLC) was performed on an Agilent 1200 series instrument equipped with photodiode array (PDA) detection (controlled by Chem Station software) and an automated injector (100 μL loop volume). Analytical separations were performed on a Nucleosil 100-5 OH (4.6×250 mm, 5 μm) analytical column at flow rates of 1.0 mL min.⁻¹. The solvent system used throughout this study was buffer A: isopropanol; buffer B: hexane. Isocratic flow of 1% isopropanol (buffer A) and 99% hexane (buffer B) was employed throughout this study.

Solvents and Reagents

Dichloromethane (DCM) was supplied by Merck and distilled over CaH prior to use. Acetic acid (AcOH), diethyl ether (Et₂O), ethyl acetate (EtOAc), hexane and methanol (MeOH) were used as supplied by Merck. (1,3-Bis-(2,4,6-trimethylphenyl)-2-imidazolidinylidene)dichloro-(o-isopropoxyphenylmethylene)ruthenium, cis-2-butene (99%), 1-butene, 1-pentene, 1-hexene, 1-heptene, 1-octene, 1-nonene, 1-decene and (S)-2-(Fmoc-amino)-4-pentenoic acid were used as supplied by Aldrich. High purity (<10 ppm oxygen) argon and hydrogen were supplied by BOC gases and additional purification was achieved by passage of the gases through water, oxygen and hydrocarbon traps.

### Example 1

### General Procedure for the Synthesis of N-Fmoc Amino Acid Analogues

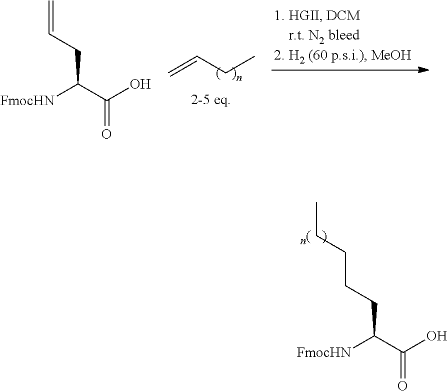

In a dry box under N₂ atmosphere, a Schlenk vessel equipped with a magnetic stir bar was charged with N-Fmoc allylglycine (100 mg, 0.296 mmol), degassed DCM (6.0 mL), terminal alkene (1.48 mmol, 5 eq.) and HGII (9.29 mg, 5 mol %). The vessel was sealed, removed from the dry box and attached to a vacuum manifold. The vessel was placed under a flow of nitrogen and the quick fit stopper replaced with a suba seal pierced with a 26 gauge needle to allow a constant flow of nitrogen over the top of the reaction. The reaction was stirred at room temperature overnight allowing all of the DCM to evaporate. The residue was washed with hexane (2×10 mL) and collected via filtration or centrifuge. The residue was then re-dissolved in MeOH (10 mL) and transferred to a Fischer-Porter tube. The vessel was charged with H₂ (60 p.s.i.), sealed and stirred at room temperature overnight. The reaction mixture was then concentrated in vacuo and the residual brown solid was purified via column chromatography to obtain pure N-Fmoc amino acid analogue. Alternatively, the residual brown solid was taken up in a small quantity of Et₂O. Insoluble matter was removed by filtration. The filtrate was concentrated in vacuo to give an off white solid.

Manual Peptide Synthesis

Manual SPPS was carried out using fritted plastic syringes, allowing filtration of solution without the loss of resin. The tap fitted syringes were attached to a vacuum tank and all washings were removed in vacuo. This involved soaking the resin in the required solvent for a reported period of time followed by evacuation to allow the removal of excess reagents before subsequent coupling reactions.

In a fritted syringe, the Rink amide resin (loading g/mol) was swollen with DCM (5 mL; 3×1 min, 1×60 min) and DMF (5 mL; 3×1 min, 1×30 min). Prior to the first coupling, the resin was subjected to Fmoc-deprotection in the presence of 20% v/v piperidine in DMF (5 mL; 1×1 min, 2×10 min) and further washed with DMF (5 mL; 5×1 min) to ensure traces of excess reagent and by-products had been removed. Amino acid pre-activation was achieved by addition of NMM (6 equiv.) to a solution of the designated protected amino acid, Fmoc-L-Xaa-OH (3 equiv.), and HATU (3 equiv.) in DMF (3 mL). The mixture was sonicated for ˜1 min and the resulting solution then added to the resin-tethered amino acid and shaken gently for a reported period of time. At the end of this reaction duration, the peptidyl-resin was washed with DMF (7 mL; 3×1 min) to ensure excess reagents were removed. Kaiser tests were performed to monitor coupling success and any incomplete coupling reactions were repeated with extended reaction times. Once a negative test for the presence of free amines was achieved, the resin-tethered peptide was deprotected with 20% v/v piperidine in DMF (5 mL; 1×1 min, 2×10 min) and further washed with DMF (5 mL; 5×1 min) to remove traces of base prior to subsequent amino acid couplings. The above procedure was repeated until the desired sequence was constructed and the resin then washed with DMF (5 mL; 3×1 min), DCM (5 mL; 3×1 min), MeOH (5 mL; 3×1 min), DCM (5 mL; 3×1 min) and MeOH (5 mL; 3×1 min), then left to dry in vacuo for 1 h. After sequence completion, the resin-tethered peptide was subjected to TFA-mediated cleavage for chromatographic and mass spectral analysis.

Compound Characterisation

While the amino acid analogues produced by the general procedure described above may be used (e.g. incorporated into a peptide sequence) without further purification, each of the following amino acid analogues were purified for by column chromatography for characterisation purposes.

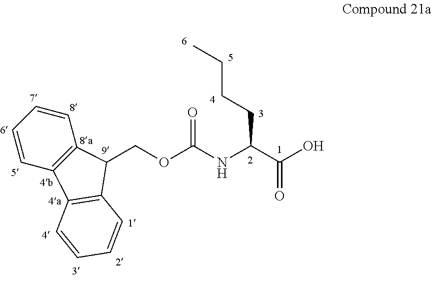 [SMILES: CCCC[C@H](NC(=O)OCC1c2ccccc2-c2ccccc21)C(=O)O]

cis-2-Butene was used as a propene equivalent. The crude reaction product was purified by column chromatography (EtOAc:Hexane:AcOH=3:1:0.05) to give the title compound as a colourless solid (105 mg, 93%), m.p. 129-130° C. vₘₐₓ (neat): 3484s, 1699s, 1674s, 1559m, 1476m, 1449m, 1390m, 1323m, 1267m, 1165m, 1088w, 1048w, 934w, 859w, 888w, 754w, 739w. ¹H n.m.r. (400 MHz, CDCl₃): δ 0.96-0.85 (m, 3H, H6), 1.44-1.23 (m, 4H, H4 & H5), 1.97-1.65 (m, 2H, H3), 4.23 (t, J=6.4 Hz, 1H, H2), 4.47-4.36 (m, 2H, CH₂), 4.49 (bs, 1H, H9′), 5.27 (bd, J=7.2 Hz, 1H, NH), 7.31 (t, J=6.8 Hz, 2H, H2′ & H7′), 7.40 (t, J=7.2 Hz, 2H, H3′ & H6′), 7.63-7.52 (m, 2H, H1′ & H8′), 7.76 (d. J 7.2H, 2H, H4′ & H5′), OH not observed. ¹³C n.m.r. (100 MHz, CDCl₃): δ 14.0 (C6), 32.2, 27.5, 22.4, 54.0, 47.4, 67.3, 127.9, 127.3, 125.3, 125.0, 120.2, 141.5, 143.9 & 144.1 (C8′a & C9′a), 156.3 (OCONH), 177.4 (C1). LR-MS: 21a t_{R}=5.21 min (>98% pure), (ESI⁺, MeOH): m/z 376.1 (M+Na)⁺, C₂₃H₂₃NNaO₄⁺ requires 376.15.

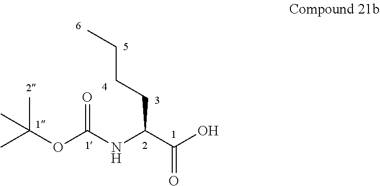 [SMILES: CCCC[C@H](NC(=O)OC(C)(C)C)C(=O)O]

Purified via column chromatography (EtOAc:Hexane:AcOH=3:1:0.05) to give the title compound as a colourless oil (111 mg, 78%). vₘₐₓ (neat): 3366s, 2960s, 2874s, 1699s, 1653s, 1507s, 1457s, 1394s, 1368s, 1249s, 1164s, 1107m, 1049m, 1021m, 851w, 799w, 738w. Mixture of rotamers observed (A:B=1:2), ¹H n.m.r. (400 MHz, CDCl₃): δ 0.90 (t, J=6.0 Hz, 3H, H6), 1.29-1.41 (m, 4H, H4 & H5), 1.44 (s, 9H, H2″), 1.58-1.94 (m, 2H, H3), 4.12 (m, 1H, H2, rotamer A), 4.30 (m, 1H, H2 rotamer B), 5.01 (bs, 1H, NH rotamer B), 6.21 (bs, 1H, NH rotamer A), 8.30 (bs, 1H, OH). ¹³C n.m.r. (100 MHz, CDCl₃): δ 13.9 (C6), 22.4 (C5), 27.5 (C4), 28.5 (C2″), 33.3 (C3), 53.6 (C2), 80.3 (C1″), 155.8 (C1′), 177.8 (C1). HRMS (ESI⁻, MeOH): m/z 230.1394 (M−H)⁻, C₁₁H₂₀NO₄⁻ requires 230.1398.

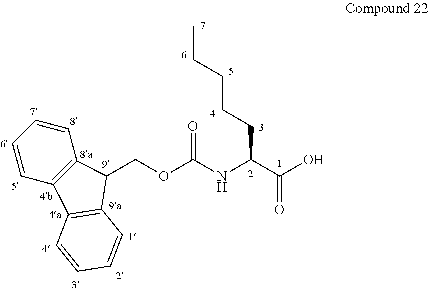 [SMILES: CCCCC[C@H](NC(=O)OCC1c2ccccc2-c2ccccc21)C(=O)O]

Purified via column chromatography (EtOAc:Hexane:AcOH=3:1:0.05) to give the title compound as a white solid (93.5 mg, 86%), m.p. 111-112° C. vₘₐₓ (neat): 3376m, 2920s, 2853s, 1751s, 1734s, 1671s, 1560s, 1457s, 1377m, 1268m, 1181m, 1167m, 1124m, 1044w, 739m, 734m. ¹H n.m.r. (400 MHz, CDCl₃): δ 0.89 (3H, t, J=6.4 Hz, H7), 1.26-1.38 (6H, m, H4-6), 1.66-1.94 (2H, m, H3), 4.23 (1H, t, J=6.8 Hz, H2), 4.38-4.45 (1H, m, CH), 4.42 (2H, d, J=6.8 Hz, CH₂), 5.26 (1H, d, J=7.6 Hz, NH), 7.31 (t, J=7.4 Hz, 2H, H2′ & H7′), 7.40 (t, J=7.4 Hz, 2H, H3′ & H6′), 7.58-7.61 (m, 2H. H1′ & H8′), 7.76 (d, J=7.4 Hz, 2H, H4′ & H5′), OH not observed. ¹³C n.m.r. (100 MHz, CDCl₃): δ 14.1 (C7), 22.5, 25.0, 31.4, 32.4, 47.3, 54.0, 67.2, 120.1, 125.2, 127.2, 127.9, 141.5, 143.8 & 144.0 (C8′a & C9′a), 156.2 (OCONH), 177.4 (C1). LC-MS: 22 t_{R}=5.55 min (>98% pure), (ESI⁺, MeOH): m/z 390.1 (M+Na)⁻, C₂₂H₂₅NO₄Na requires 390.17.

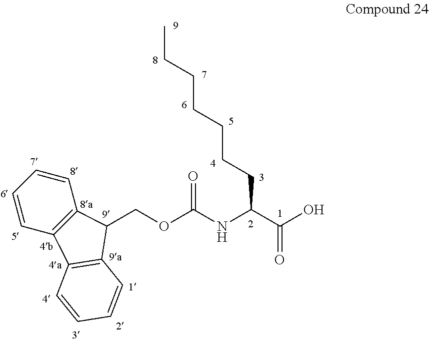 [SMILES: CCCCCCC[C@H](NC(=O)OCC1c2ccccc2-c2ccccc21)C(=O)O]

Purified via column chromatography (EtOAc:Hexane:AcOH=3:1:0.05) to give the title compound as a colourless solid (73.4 mg, 65%), m.p. 123-124° C. vₘₐₓ (neat): 3374m, 2923s, 2856s, 1754s, 1739m, 1675s, 1558s, 1456s, 1377m, 1266m, 1165m, 1125m, 1088w, 1046w, 741m, 734m cm⁻¹. ¹H n.m.r. (400 MHz, CDCl₃): δ 0.88 (3H, t, J=6.8 Hz, H8), 1.29-1.43 (8H, m, H4-7), 1.66-1.94 (2H, m, H3), 4.23 (1H, t, J=6.8 Hz, H2), 4.38-4.45 (1H, m, CH), 4.42 (2H, d, J=6.8 Hz, CH₂), 5.26 (1H, d, J=8.0 Hz, NH), 7.31 (t, J=7.4 Hz, 2H, H2′ & H7′), 7.40 (t, J=7.4 Hz, 2H, H3′ & H6′), 7.58-7.61 (m, 2H, H1′ & H8′), 7.76 (d, J=7.4 Hz, 2H, H4′ & H5′), OH not observed. ¹³C n.m.r. (100 MHz, CDCl₃): δ 14.2 (C8), 22.7, 25.3, 28.9, 31.7, 32.5, 47.3, 54.0, 67.2, 120.1, 125.2, 127.2, 127.9, 141.5, 143.9 & 144.0 (C8′a & C9′a), 156.2 (OCONH), 177.4 (C1). LC-MS: 23 t_{R}=5.23 min (>98% pure), (ESI⁺, MeOH): m/z 404.1 (M+Na)⁺, C₂₃H₂₇NO₄Na requires 404.18.

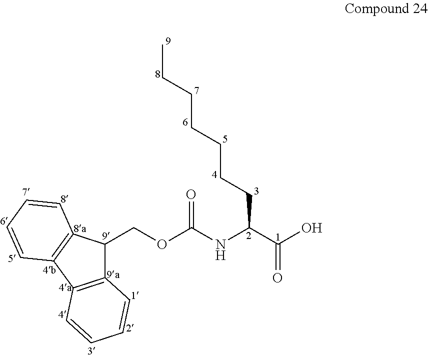 [SMILES: CCCCCCC[C@H](NC(=O)OCC1c2ccccc2-c2ccccc21)C(=O)O]

Purified via column chromatography (EtOAc:Hexane:AcOH=3:1:0.05) to give the title compound as a colourless solid (94.8 mg, 81%), m.p. 112-114° C. vₘₐₓ (neat): 3323bm, 2928s, 2856s, 1716s, 1635m, 1519s, 1450s, 1418m, 1338m, 1264m, 1115w, 1078m, 1050m, 758m, 739s. ¹H n.m.r. (400 MHz, CDCl₃): δ 0.88 (3H, t, J=6.8 Hz, H9), 1.26-1.42 (10H, m, H4-8), 1.64-1.94 (2H, m, H3), 4.22 (1H, t, J=6.8 Hz, H2), 4.38-4.45 (1H, m, CH), 4.41 (2H, d, J=7.2 Hz, CH₂), 5.26 (1H, d, J=8.0 Hz, NH), 7.30 (t, J=7.4 Hz, 2H, H2′ & H7′), 7.39 (t, J=7.4 Hz, 2H, H3′ & H6′), 7.57-7.60 (m, 2H, H1′ & H8′), 7.75 (d, J=7.4 Hz, 2H, H4′ & H5′), OH not observed. ¹³C n.m.r. (100 MHz, CDCl₃): δ 14.2 (C9), 22.7, 25.4, 29.2, 29.3, 31.9, 32.5, 47.3, 54.0, 67.2, 120.1, 125.2, 127.2, 127.9, 141.5, 143.9 & 144.0 (C8′a & C9′a), 156.3 (OCONH), 177.2 (C1). LC-MS: 24 t_{R}=5.49 min (>98% pure), (ESI⁺, MeOH): m/z 418.1 (M+Na)⁺, C₂₄H₂₉NO₄Na requires 418.20.

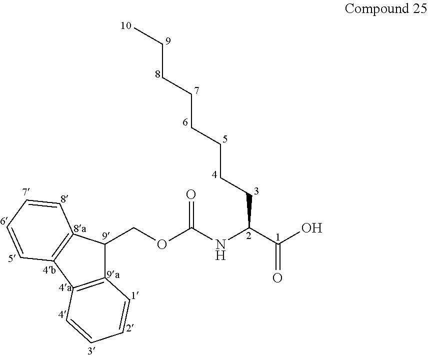 [SMILES: CCCCCCCC[C@H](NC(=O)OCC1c2ccccc2-c2ccccc21)C(=O)O]

Purified via column chromatography (EtOAc:Hexane:AcOH=3:1:0.05) to give the title compound as a colourless solid (88.5 mg, 73%), m.p. 65-67° C. vₘₐₓ (neat): 3431bm, 3065m, 2925s, 2854s, 1718s, 1696s, 1539m, 1517m, 1450s, 1419w, 1340m, 1248m, 1115w, 1052m, 758m, 738s. ¹H n.m.r. (400 MHz, CDCl₃): δ 0.88 (3H, t, J=6.8 Hz, H10), 1.26-1.37 (12H, m, H4-9), 1.64-1.93 (2H, m, H3), 4.22 (1H, t, J=6.8 Hz, H2), 4.37-4.48 (1H, m, CH), 4.41 (2H, d, J=6.8 Hz, CH₂), 5.30 (1H, d, J=8.4 Hz, NH), 7.30 (t, J=7.4 Hz, 2H, H2′ & H7′), 7.39 (t, J=7.4 Hz, 2H, H3′ & H6′), 7.57-7.60 (m, 2H, H1′ & H8′), 7.75 (d, J=7.4 Hz, 2H, H4′ & H5), OH not observed. ¹³C n.m.r. (100 MHz, CDCl₃): δ 14.2 (C10), 22.8, 25.4, 29.31, 29.35, 29.5, 32.0, 32.5, 47.3, 54.0, 67.2, 120.1, 125.2, 127.2, 127.9, 141.5, 143.9 & 144.0 (C8′a & C9′a), 156.3 (OCONH), 177.5 (C1). LC-MS: 25 t_{R}=5.48 min (>98% pure), ESI⁺, MeOH): m/z 432.1 (M+Na)⁺, C₂₅H₃₁NO₄Na requires 432.22.

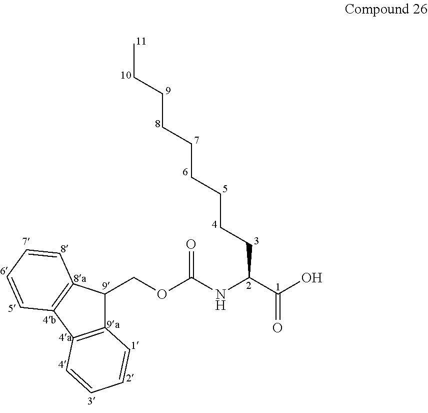 [SMILES: CCCCCCCCC[C@H](NC(=O)OCC1c2ccccc2-c2ccccc21)C(=O)O]

Purified via column chromatography (EtOAc:Hexane:AcOH=3:1:0.05) to give the title compound as a colourless solid (95.3 mg, 76%), m.p. 96-98° C. vₘₐₓ (neat): 3417bm, 2926s, 2854s, 1717s, 1647s, 1559m, 1517m, 1450m, 1419w, 1339m, 1247m, 1105w, 1051w, 758m, 738m. ¹H n.m.r. (400 MHz, CDCl₃): δ 0.88 (3H, t, J=6.8 Hz, H11), 1.26-1.32 (14H, m, H4-10), 1.61-1.91 (2H, m, H3), 4.23 (1H, t, J=6.8 Hz, H2), 4.40-4.47 (3H, m, CH & CH₂), 5.25 (1H, d, J=8.0 Hz, NH), 7.31 (t, J=7.4 Hz, 2H, H2′ & H7′), 7.40 (t, J=7.4 Hz, 2H, H3′ & H6′), 7.59-7.60 (m, 2H, H1′ & H8′), 7.76 (d, J=7.4 Hz, 2H, H4′ & H5′), OH not observed. ¹³C n.m.r. (100 MHz, CDCl₃): δ 14.2 (C12), 22.8, 25.4, 29.3, 29.4, 29.5, 29.6, 32.0, 32.5, 47.3, 53.9, 67.2, 120.1, 125.2, 127.2, 127.9, 141.5, 143.9 & 144.0 (C8′a & C9′a), 156.2 (OCONH), 177.4 (C1). LC-MS: 26 t_{R}=5.47 min (>98% pure), ESI⁺, MeOH): m/z 446.1 (M+Na)⁺, C₂₆H₃₃NO₄Na requires 446.23.

The reaction was also performed on larger scale following the general procedure described in Section 1.3. N-Fmoc allylglycine (1.00 g, 2.96 mmol), degassed DCM (30 mL), 1-octene (2.32 mL, 14.8 mmol, 5 eq.) and HGII (46.5 mg, 2.5 mol %) were used. Purification of the crude product was performed as described in Section 1.3 to give the title compound (0.89 g, 71%). Spectroscopic data was consistent with previously described data for 26.

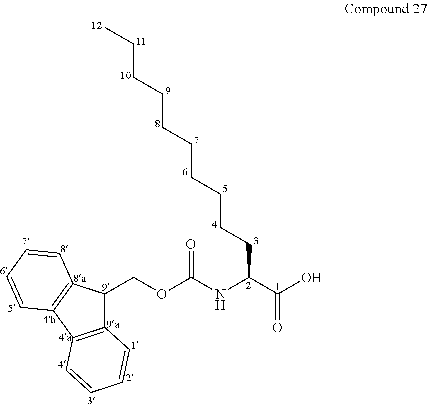 [SMILES: CCCCCCCCCC[C@H](NC(=O)OCC1c2ccccc2-c2ccccc21)C(=O)O]

Purified via column chromatography (EtOAc:Hexane:AcOH=3:1:0.05) to give the title compound as a colourless solid (103.6 mg, 80%), m.p. 61-62° C. vₘₐₓ (neat): 3334bm, 2924s, 2853s, 1715s, 1635m, 1521m, 1465m, 1450s, 1419m, 1339m, 1247m, 1117w, 1079m, 1052m, 758m, 738m. ¹H n.m.r. (400 MHz, CDCl₃): δ 0.88 (3H, t, J=6.8 Hz, H12), 1.26-1.37 (16H, m, H4-11), 1.64-1.93 (2H, m, H3), 4.22 (1H, t, J=6.8 Hz, H2), 4.37-4.48 (1H, m, CH), 4.41 (2H, d, J=6.8 Hz, CH₂), 5.31 (1H, d, J=8.0 Hz, NH), 7.30 (t, J=7.4 Hz, 2H, H2′ & H7′), 7.39 (t, J=7.4 Hz, 2H, H3′ & H6′), 7.57-7.60 (m, 2H, H1′ & H8′), 7.75 (d, J=7.4 Hz, 2H, H4′ & H5′), OH not observed. ¹³C n.m.r. (100 MHz, CDCl₃): δ 14.2 (C12), 22.8, 25.4, 29.3, 29.46, 29.54, 29.71, 29.72, 32.0, 32.5, 47.3, 54.1, 67.2, 120.1, 125.2, 127.2, 127.9, 141.5, 143.8 & 144.0 (C8′a & C9′a), 156.3 (OCONH), 177.6 (C1). LC-MS: 27 t_{R}=5.45 min (>98% pure), ESI⁺, MeOH): m/z 460.2 (M+Na)⁺, C₂₇H₃₅NO₄Na requires 460.25.

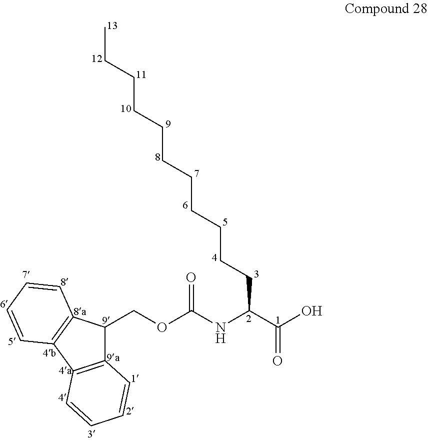 [SMILES: CCCCCCCCCCC[C@H](NC(=O)OCC1c2ccccc2-c2ccccc21)C(=O)O]

Purified via column chromatography (EtOAc:Hexane:AcOH=3:1:0.05) to give the title compound as a colourless solid (100.2 mg, 75%), m.p. 95-96° C. vₘₐₓ(neat): 3339m, 3065m, 2925s, 2852s, 1718s, 1696s, 1539m, 1517m, 1465m, 1450m, 1419w, 1340m, 1247m, 1079w, 1052w, 758m, 739s. ¹H n.m.r. (400 MHz, CDCl₃): δ 0.89 (3H, t, J=6.8 Hz, H13), 1.26-1.37 (18H, m, H4-12), 1.62-1.93 (2H, m, H3), 4.22 (1H, t, J=6.8 Hz, H2), 4.38-4.48 (1H, m. CH), 4.41 (2H, d, J=6.8 Hz, CH₂), 5.30 (1H, d, J=8.0 Hz, NH), 7.30 (t, J=7.4 Hz, 2H, H2′ & H7′), 7.39 (t, J=7.4 Hz, 2H, H3′ & H6′), 7.58-7.61 (m, 2H, H1′ & H8′), 7.76 (d, J=7.4 Hz, 2H, H4′ & H5′), OH not observed. ¹³C n.m.r. (100 MHz, CDCl₃): δ 14.2 (C13), 22.8, 25.4, 29.3, 29.49, 29.54, 29.71, 29.75, 29.76, 32.1, 32.5, 47.3, 54.0, 67.2, 120.1, 125.2, 127.2, 127.9, 141.5, 143.8 & 144.0 (C8′a & C9′a), 156.3 (OCONH), 177.6 (C1). LC-MS: 28 t_{R}=5.44 min (>98% pure), (ESI⁺, MeOH): m/z 474.1 (M+Na)⁺, C₂₈H₃₇NO₄Na requires 474.26.

Compound 29

The manual peptide synthesis procedure outlined in the General Experimental Section was used for the synthesis of peptide 29 on Fmoc-Rink amide resin (250 mg, 0.10 mmol). Quantities of HATU, NMM, piperidine and each Fmoc-amino acid were used as described by the protocol and kept constant throughout the synthesis. The total amount of each coupling reagent and successive amino acid required, along with their reaction duration, is summarised in the table below:

| | Mass (mg) or | | Reaction |
|---|---|---|---|
| Reagent | Volume (μL) | | Time (min) |
| HATU | 76.0 | mg | — |
| NMM | 66 | μL | — |
| Fmoc-L-Ser(^{t}Bu)-OH | 115 | mg | 120 |
| Fmoc-L-Ala-OH | 93.4 | mg | 120 |
| Fmoc-L-Laa-OH 26 | 127 | mg | 120 |
| Fmoc-L-Thr(^{t}Bu)-OH | 119 | mg | 120 |
| Fmoc-L-His(Boc)-OH | 186 | mg | 120 |

After sequence completion, the resin-bound peptide was transferred into a fritted syringe and washed with DMF (5 mL; 3×1 min), DCM (5 mL; 3×1 min), MeOH (5 mL; 3×1 min), DCM (5 mL; 3×1 min) and MeOH (5 mL; 3×1 min), then left to dry in vacuo for 1 h. The resin-tethered peptide was subjected to TFA-mediated cleavage for RP-HPLC and mass spectral analysis. This supported formation of the desired peptide 29. Mass spectrum (ESI⁺, MeCN:H₂O:HCOOH): m/z 597.3 [M+H]⁺, C₂₇H₄₉N₈O₇ requires 597.37. RP-HPLC (Agilent: Vydac C18 analytical column, 15→45% buffer B over 30 min): t_{R}=16.3 min.

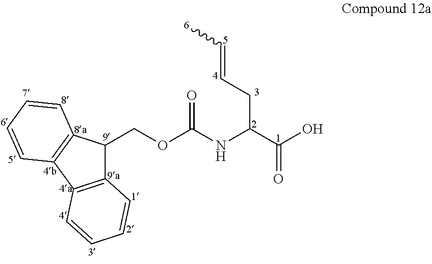

¹H n.m.r. (400 MHz, CDCl₃): δ 1.66 (d, J=6.2 Hz, 3H, H6), 2.39-2.63 (m, 2H, H3), 4.08-4.27 (m, 1H, H2), 4.36-4.55 (in, 3H, CH₂ & H9′), 5.26-5.37 (m, 2H, H4 & NH), 5.52-5.62 (m, 1H, H5), 7.31 (t, J=7.3 Hz, 2H, H2′ & H7′), 7.40 (t, J=7.4 Hz, 2H, H3′ & H6′) 7.54-7.64 (m, 2H, H1′ & H8′), 7.77 (d, J=7.4 Hz, 2H, H4′ & H5′), 8.81 (bs, 1H, OH). LRMS: (ESI⁺, MeOH): m/z (M+Na)⁺ 374.1, C₂₁H₂₁NO₄Na requires 374.14.

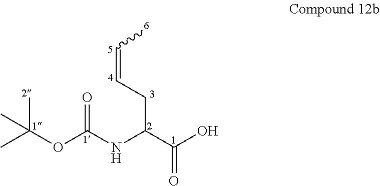

¹H n.m.r. (400 MHz, CDCl₃): δ 1.44 (s, 9H, H2″), 1.67 (d, J=6.2 Hz, 3H, H6), 2.34-2.59 (m, 2H, H3), 4.02-4.45 (m, 1H, H2), 4.99 (bs, 1H, NH), 5.26-5.73 (m, 2H, H4 & H5), OH not observed. LR-MS: (ESI⁺, MeOH): m/z 252.1 (M+Na)⁺, C₁₁H₁₉NNaO₄⁺ requires 252.12.

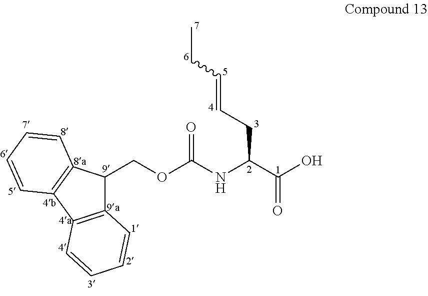 [SMILES: CCC=CC[C@H](NC(=O)OCC1c2ccccc2-c2ccccc21)C(=O)O]

¹H n.m.r. (400 MHz, CDCl₃): δ 0.98 (3H, t, J=6.8 Hz, H7), 2.02-2.10 (2H, m, H6), 2.40-2.65 (2H, m, H3), 4.13-4.23 (1H, m, H2), 4.40-4.55 (3H, m, CH & CH₂), 5.17 (1H, d, J=7.6 Hz, NH), 5.26-5.35 (1H, m, H5), 5.55-5.65 (1H, m, H4), 7.29 (t, J=7.4 Hz, 2H, H2′ & H7′), 7.40 (t, J=7.4 Hz, 2H, H3′ & H6′), 7.58-7.61 (m, 2H, H1′ & H8′), 7.76 (d, J=7.4 Hz, 2H, H4′ & H5′), OH not observed. LRMS: (ESI⁺, MeOH): m/z 388.1 (M+Na)⁺, C₂₂H₂₃NO₄Na requires 388.15.

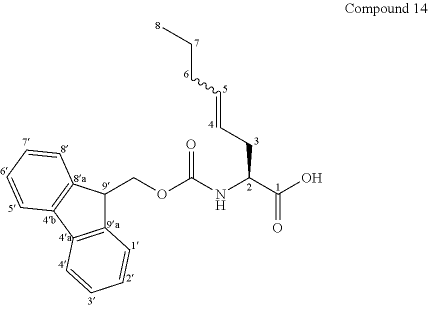 [SMILES: CCCC=CC[C@H](NC(=O)OCC1c2ccccc2-c2ccccc21)C(=O)O]

¹H n.m.r. (400 MHz, CDCl₃): δ 0.98 (3H, t, J=6.8 Hz, H8), 1.70-1.74 (2H, m, H7), 2.02-2.10 (2H, m, H6), 2.40-2.65 (2H, m, H3), 4.13-4.23 (1H, m, H2), 4.40-4.55 (3H, m, CH & CH₂), 5.17 (1H, d, J=7.6 Hz, NH), 5.26-5.35 (1H, m, H5), 5.55-5.65 (1H, m, H4), 7.29 (t, J=7.4 Hz, 2H, H2′ & H7′), 7.40 (t, J=7.4 Hz, 2H, H3′ & H6′), 7.58-7.61 (m, 2H, H1′ & H8′), 7.76 (d, J=7.4 Hz, 2H, H4′ & H5′), OH not observed. LRMS: (ESI⁺, MeOH): m/z 401.9 (M+Na)⁺, C₂₃H₂₅NO₄Na requires 402.17.

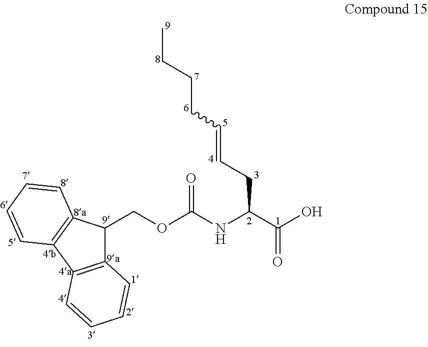 [SMILES: CCCCC=CC[C@H](NC(=O)OCC1c2ccccc2-c2ccccc21)C(=O)O]

¹H n.m.r. (400 MHz, CDCl₃): δ 0.90 (3H, t, J=6.8 Hz, H9), 1.33-1.38 (4H, m, H7 & 8), 1.99-2.06 (2H, m, H6), 2.42-2.56 (2H, m, H3), 4.14-4.24 (1H, m, H2), 4.41-4.56 (3H, m, CH & CH₂), 5.29-5.35 (2H, m, H5 & NH), 5.57-5.62 (1H, m, H4), 7.29 (t, J=7.4 Hz, 2H, H2′ & H7′), 7.40 (t, J=7.4 Hz, 2H, H3′ & H6′), 7.58-7.61 (m, 2H, H1′ & H8′), 7.76 (d, J=7.4 Hz, 2H, H4′ & H5′), 10.22 (1H, br s, OH). LRMS: (ESI⁺, MeOH): m/z 416.1 (M+Na)⁺, C₂₄H₂₇NO₄Na requires 416.18.

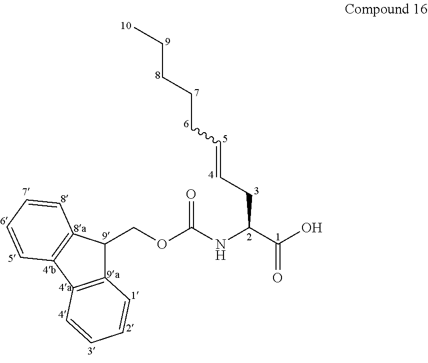 [SMILES: CCCCCC=CC[C@H](NC(=O)OCC1c2ccccc2-c2ccccc21)C(=O)O]

¹H n.m.r. (400 MHz, CDCl₃): δ 0.88 (3H, t, J=6.8 Hz, H10), 1.23-1.38 (6H, m, H7-9), 1.96-2.06 (2H, m, H6), 2.46-2.61 (2H, m, H3), 4.16-4.26 (1H, m, H2), 4.34-4.54 (3H, m, CH & CH₂), 5.29-5.34 (2H, m, H5 & NH), 5.56-5.64 (1H, m, H4), 7.29 (t, J=7.4 Hz, 2H, H2′ & H7′), 7.40 (t, J=7.4 Hz, 2H, H3′ & H6′), 7.58-7.61 (m, 2H, H1′ & H8′), 7.76 (d, J=7.4 Hz, 2H, H4′ & H5′), 9.65 (1H, br s, OH). LRMS: (ESI⁺, MeOH): m/z 430.1 (M+Na)⁺, C₂₅H₂₉NO₄Na requires 430.20.

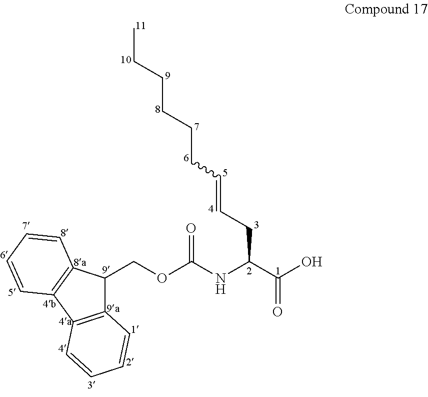 [SMILES: CCCCCCC=CC[C@H](NC(=O)OCC1c2ccccc2-c2ccccc21)C(=O)O]

¹H n.m.r. (400 MHz, CDCl₃): δ 0.90 (3H, t, J=6.8 Hz, H11), 1.27-1.38 (8H, m, H7-10), 1.96-2.06 (2H, m, H6), 2.48-2.61 (2H, m, H3), 4.19-4.24 (1H, m, H2), 4.41-4.48 (3H, m, CH & CH₂), 5.29-5.39 (2H, m, H5 & NH), 5.58-5.61 (1H, m, H4), 7.29 (t, J=7.4 Hz, 2H, H2′ & H7′), 7.40 (t, J=7.4 Hz, 2H, H3′ & H6′), 7.58-7.61 (m, 2H, H1′ & H8′), 7.76 (d, J=7.4 Hz, 2H, H4′ & H5′), 9.82 (1H, br s, OH). LRMS: (ESI⁺, MeOH): m/z 444.1 (M+Na)⁺, C₂₆H₃₁NO₄Na requires 444.21.

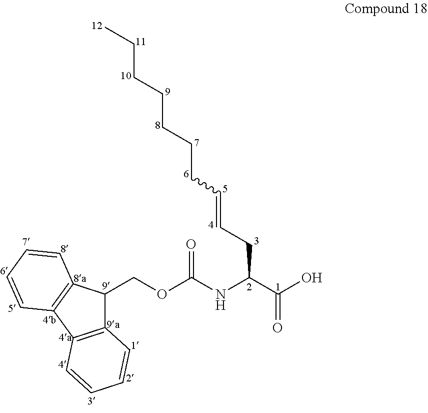 [SMILES: CCCCCCCC=CC[C@H](NC(=O)OCC1c2ccccc2-c2ccccc21)C(=O)O]

¹H n.m.r. (400 MHz, CDCl₃): δ 0.90 (3H, t, J=6.8 Hz, H12), 1.27-1.38 (10H, m, H7-11), 1.96-2.06 (2H, m, H6), 2.44-2.61 (2H, m, H3), 4.19-4.24 (1H, m, H2), 4.41-4.48 (3H, m, CH & CH₂), 5.27-5.39 (2H, m, H5 & NH), 5.58-5.62 (1H, m, H4), 7.29 (t, J=7.4 Hz, 2H, H2′ & H7′), 7.40 (t, J=7.4 Hz, 2H, H3′ & H6′), 7.58-7.61 (m, 2H, H1′ & H8′), 7.76 (d, J=7.4 Hz, 2H, H4′ & H5′), 9.98 (1H, br s, OH). (ESI⁺, MeOH): m/z 458.1 (M+Na)⁺, C₂₇H₃₃NO₄Na requires 458.23.

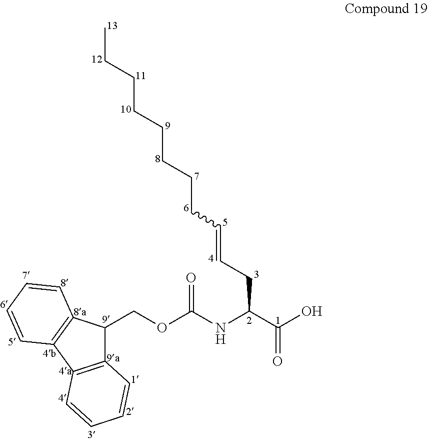 [SMILES: CCCCCCCCC=CC[C@H](NC(=O)OCC1c2ccccc2-c2ccccc21)C(=O)O]

¹H n.m.r. (400 MHz, CDCl₃): δ 0.90 (3H, t, J=6.8 Hz, H13), 1.26-1.38 (12H, m, H7-12), 1.96-2.08 (2H, m, H6), 2.48-2.61 (2H, m, H3), 4.19-4.24 (1H, m, H2), 4.38-4.48 (3H, m, CH & CH₂), 5.30-5.39 (2H, m, H5 & NH), 5.55-5.62 (1H, m, H4), 7.29 (t, J=7.4 Hz, 2H, H2′ & H7′), 7.40 (t, J=7.4 Hz, 2H, H3′ & H6′), 7.58-7.61 (m, 2H, H1′ & H8′), 7.76 (d, J=7.4 Hz, 2H, H4′ & H5′), 10.16 (1H, br s, OH). (ESI⁺, MeOH): m/z 472.0 (M+Na)⁺, C₂₈H₃₅NO₄Na requires 472.25.

### Example 2

### General Procedure for the Incorporation of an Effector Molecule into the Amino Acid Analogue

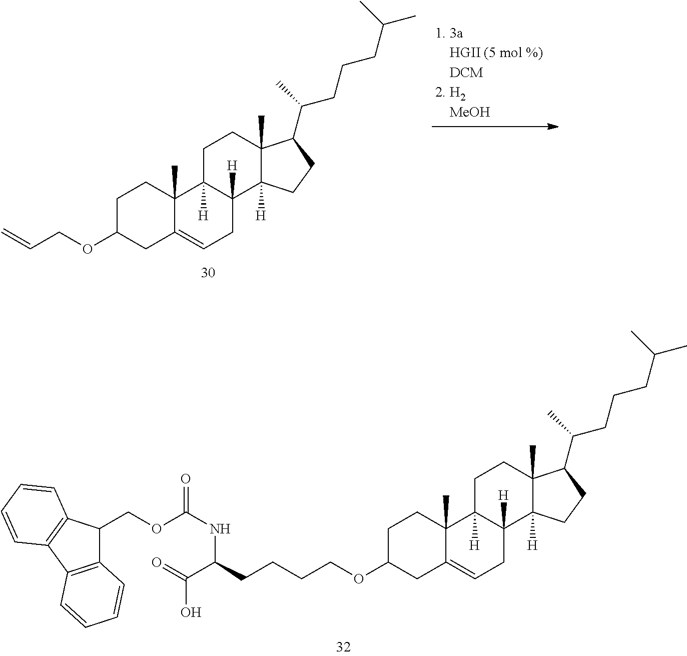

In a dry box under N₂ atmosphere, a Schlenk vessel equipped with a magnetic stir bar was charged with N-Fmoc allylglycine (92 mg, 0.27 mmol), O-allylcholesterol 30 (463 mg, 1.09 mmol, 4 eq.) degassed DCM (6.0 mL) and HGII (8.56 mg, 5 mol %). The vessel was sealed, removed from the dry box and attached to a vacuum manifold. The vessel was placed under a flow of nitrogen and the quick fit stopper replaced with a suba seal pierced with a 26 gauge needle to allow a constant flow of nitrogen over the top of the reaction. The reaction was stirred at room temperature overnight allowing all of the DCM to evaporate. The residue was washed with hexane (2×10 mL) and collected via filtration or centrifuge. The intermediate alkene 31 was reduced with high purity hydrogen in methanol to generate 32. The residue was then re-dissolved in MeOH (10 mL) and transferred to a Fischer-Porter tube. The vessel was charged with H₂ (60 p.s.i.), sealed and stirred at room temperature overnight. The crude reaction mixture was purified via column chromatography (EtOAc:Hexane:AcOH=3:1:0.05) to give the target compound 32 as a colourless solid (100 mg, 50%).

Compound Characterisation

While the amino acid analogues produced by the general procedure described above may be used (e.g. incorporated into a peptide sequence) without further purification, each of the following amino acid analogues were purified for by column chromatography for characterisation purposes.

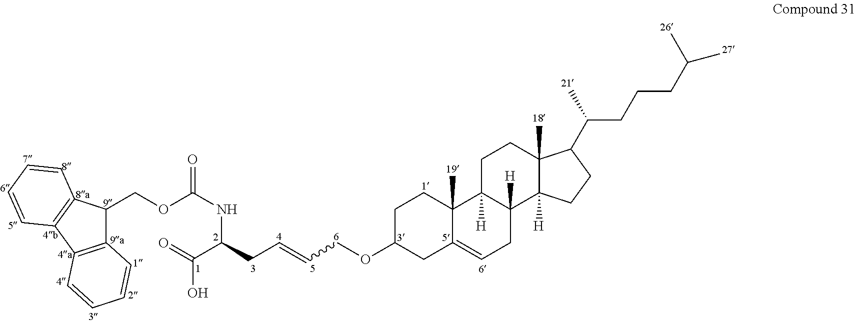

LRMS (ESI⁺, MeOH): m/z 758.2 (M+Na)⁺, C₄₈H₆₅NO₅Na⁺ requires 758.48.

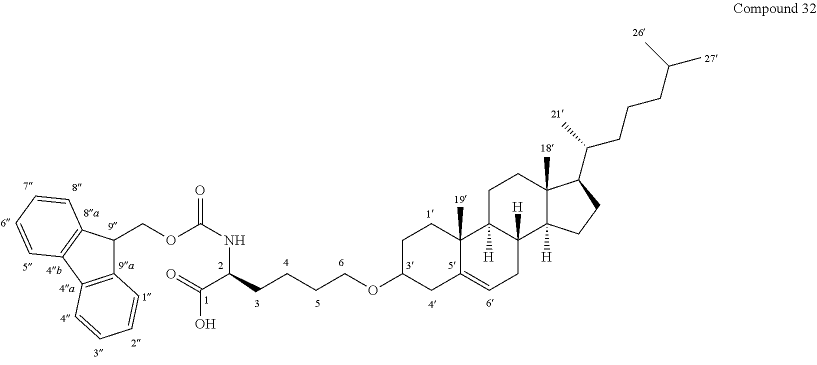

m.p. 68-72° C. vₘₐₓ (neat): 3438bs, 2942s, 2866s, 1718s, 1696s, 1516m, 1465m, 1450m, 1419w, 1379w, 1340w, 1255m, 1261m, 1104m, 1082m, 1059m, 908s, 758m, 734s cm⁻¹. ¹H n.m.r. (400 MHz, CDCl₃): δ 0.67 (s, 3H, CH₃), 0.87 (d, J=1.6 Hz, 3H, H26′ or H27′), 0.88 (d, J=1.6 Hz, 3H, H26′ or H27′), 0.92 (d, J=6.4 Hz, 3H, H21′), 0.98 (s, 3H, CH₃), 1.03-2.02 (m, 32H), 2.13-2.39 (m, 2H, H4′), 3.10-3.16 (m, 1H, H3′), 3.40-3.50 (m, 2H, H6), 4.22 (t, J=6.8 Hz, 1H, H2), 4.40 (d, J=5.6 Hz, 2H, CH₂), 4.45-4.50 (m, 1H, H9″), 5.32-5.33 (m, 1H, H6′), 5.48 (d, J=7.6 Hz, 1H, NH), 7.30 (t, J=7.4 Hz, 2H, H2″ & H7″), 7.39 (t, J=7.4 Hz, 2H, H3″ & H6″), 7.58-7.59 (m, 2H, H1″ & H8″), 7.75 (d, J=7.4 Hz, 2H, H4″ & H5″), OH not observed. ¹³C n.m.r. (100 MHz, CDCl₃): δ 12.0, 18.9, 19.5, 21.2, 22.3, 22.7, 22.9, 24.0, 24.4, 28.2, 28.4, 28.5, 29.6, 32.0, 32.08, 32.13, 35.9, 36.4, 37.0, 37.4, 39.2, 39.7, 40.0, 42.5, 47.3, 50.4, 54.0, 56.4, 56.9, 67.3, 67.7, 79.4, 120.1, 121.7, 125.2, 127.2, 127.9, 141.0, 141.5, 143.9 & 144.0 (C8′a & C9′a), 156.3 (OCONH), 176.4 (C1). HRMS (ESI⁻, MeOH): m/z 736.4955 (M−H)⁻, C₄₈H₆₆NO₅⁻ requires 736.4946.

### Example 3

### General Procedure for the Incorporation of an N-Heterocycle (e.g. Chelate for Radiolabelling) Effector Molecule into the Amino Acid Analogue

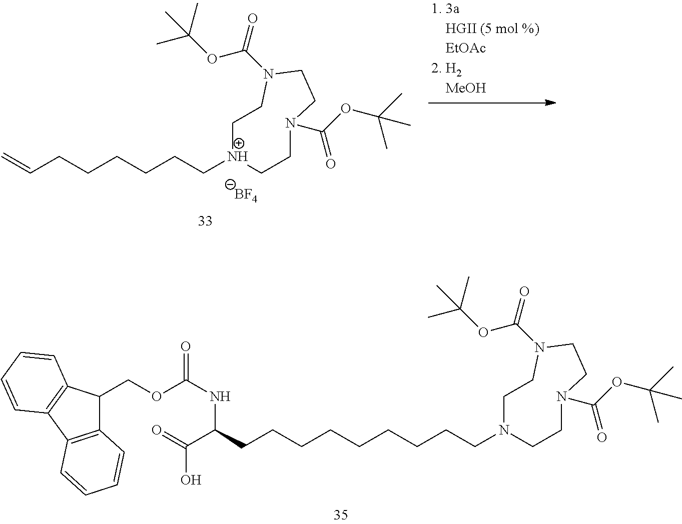

In a dry box under N₂ atmosphere, a Schlenk vessel equipped with a magnetic stir bar was charged with N-Fmoc allylglycine (41 mg, 0.122 mmol), degassed EtOAc (4.0 mL), 33 (321 mg, 0.61 mmol, 5 eq.) and HGII (3.8 mg, 5 mol %). The vessel was sealed, removed from the dry box and attached to a vacuum manifold. The vessel was placed under a flow of nitrogen and the quick fit stopper replaced with a suba seal pierced with a 26 gauge needle to allow a constant flow of nitrogen over the top of the reaction. The reaction was stirred at room temperature overnight allowing all of the DCM to evaporate. The residue was washed with hexane (2×10 mL) and collected via filtration or centrifuge. The intermediate alkene 34 was reduced with high purity hydrogen in methanol to generate 35. The residue was then re-dissolved in MeOH (10 mL) and transferred to a Fischer-Porter tube. The vessel was charged with H₂ (60 p.s.i.), sealed and stirred at room temperature overnight. The crude reaction mixture was purified via column chromatography (CHCl₃:MeOH:AcOH=1:0.05:0.05) to give the title compound as a clear film (5.8 mg, 6.3%).

Compound Characterisation

While the amino acid analogues produced by the general procedure described above may be used (e.g. incorporated into a peptide sequence) without further purification, each of the following amino acid analogues were purified for by column chromatography for characterisation purposes.

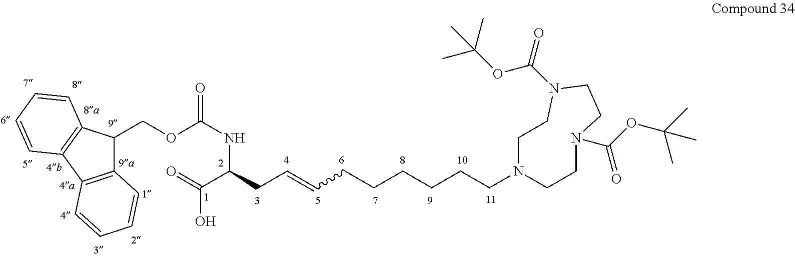 [SMILES: CC(C)(C)OC(=O)N1CCN(CCCCCCC=CC[C@H](NC(=O)OCC2c3ccccc3-c3ccccc32)C(=O)O)CCN(C(=O)OC(C)(C)C)CC1]

HRMS (ESI⁺, MeOH): m/z 749.4481 (M+H)⁺, C₄₂H₆₁N₄O₈⁺ requires 749.4484.

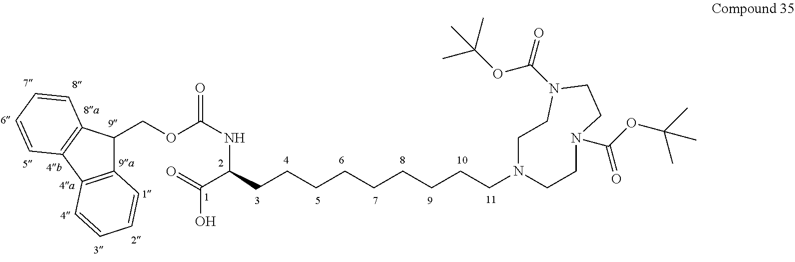 [SMILES: CC(C)(C)OC(=O)N1CCN(CCCCCCCCC[C@H](NC(=O)OCC2c3ccccc3-c3ccccc32)C(=O)O)CCN(C(=O)OC(C)(C)C)CC1]

¹H n.m.r. (400 MHz, CDCl₃): δ 1.22-1.37 (m, 14H, H4-10), 1.47 (s, 18H, 6×CH₃), 1.76-1.99 (m, 2H, H3), 2.45-2.65 (m, 4H, 2×CH₂), 2.95-3.15 (m, 6H, H11 & 2×CH₂), 3.46 (d, J=13.2 Hz, 4H, 2×CH₂), 4.22 (d, J=6.8 Hz, 2H, CH₂), 4.34-4.42 (m, 2H, H2 & H9″), 5.84 (br s, 1H, NH), 7.30 (t, J=7.4 Hz, 2H, H2″ & H7″), 7.39 (t, J=7.4 Hz, 2H, H3″ & H6″), 7.62 (d, J=7.4 Hz, 2H, H1″ & H8″), 7.75 (d, J=7.4 Hz, 2H, H4″ & H5″), OH not observed. HRMS (ESI⁺, MeOH): m/z 751.4643 (M+H)⁺, C₄₂H₆₃N₄O₈⁺ requires 751.4640.

### Example 4

### General Procedure for the Incorporation of a Polyethylene Glycol (PEG) Chain (e.g. for Water Solubility) into the Amino Acid Analogue

In a dry box under N₂ atmosphere, a Schlenk vessel equipped with a magnetic stir bar was charged with Fmoc allylglycine (100 mg, 0.30 mmol), degassed DCM (6.0 mL), 36 (300 mg, 1.48 mmol, 5 eq.) and HGII (9.3 mg, 5 mol %). The vessel was sealed, removed from the dry box and attached to a vacuum manifold. The vessel was placed under a flow of nitrogen and the quick fit stopper replaced with a suba seal pierced with a 26 gauge needle to allow a constant flow of nitrogen over the top of the reaction. The reaction was stirred at room temperature overnight allowing all of the DCM to evaporate. The residue was washed with hexane (2×10 mL) and collected via filtration or centrifuge. The intermediate alkene 37 was reduced with high purity hydrogen in methanol to generate 38. The residue was then re-dissolved in MeOH (10 mL) and transferred to a Fischer-Porter tube. The vessel was charged with H₂ (60 p.s.i.), sealed and stirred at room temperature overnight. The crude reaction mixture was purified via column chromatography to give the title compound.

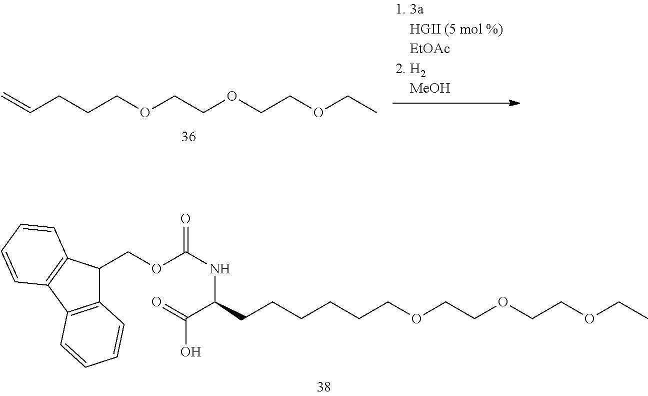 [SMILES: C=CCCCOCCOCCOCC ; CCOCCOCCOCCCCCC[C@H](NC(=O)OCC1c2ccccc2-c2ccccc21)C(=O)O]

Compound Characterisation

While the amino acid analogues produced by the general procedure described above may be used (e.g. incorporated into a peptide sequence) without further purification, each of the following amino acid analogues were purified for by column chromatography for characterisation purposes.

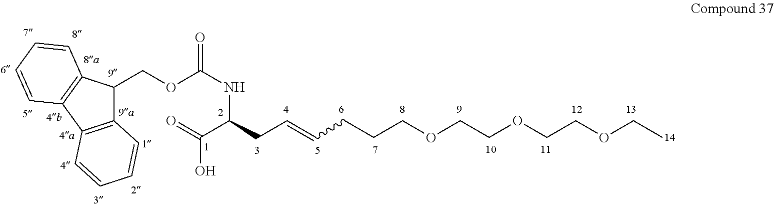 [SMILES: CCOCCOCCOCCCC=CC[C@H](NC(=O)OCC1c2ccccc2-c2ccccc21)C(=O)O]

¹H n.m.r. (300 MHz, CDCl₃): δ 1.20 (t, J=6.9 Hz, 3H, H14), 1.64 (p, J=6.3 Hz, 2H, H7), 2.07 (p, J=6.6 Hz, 2H, H6), 2.45-2.64 (m, 2H, H3), 3.45 (t, J=6.6 Hz, 2H, H8), 3.51-3.68 (m, 10H, H9-H13), 4.22 (t, J=6.6 Hz, 1H, H9″), 4.40 (d, J=6.6 Hz, 2H, CH₂), 4.36-4.53 (m, 1H, H2), 5.26-5.71 (m, 3H, H4, H5 & NH), 7.31 (t, J=7.2 Hz, 2H, H2″ & H7″), 7.39 (t, J=7.2 Hz, 2H, H3″ & H6″), 7.60 (d, J=6.6 Hz, 2H, H1″ & H8″), 7.74 (d, J=7.2 Hz, 2H, H4″ & H5″), OH not observed. LRMS (ESI⁺, MeOH): m/z 534.1 (M+Na)⁺, C₂₉H₃₇NO₇Na⁺ requires 534.2.

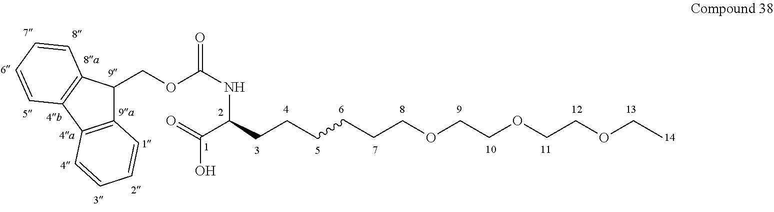 [SMILES: CCOCCOCCOCCCCCC[C@H](NC(=O)OCC1c2ccccc2-c2ccccc21)C(=O)O]

¹H n.m.r. (400 MHz, CDCl₃): δ 1.20 (t, J=6.9 Hz, 3H, H14), 1.24-1.45 (m, 6H, H4-H6), 1.48-1.64 (m, 2H, H7), 1.64-1.96 (m, 2H, H3), 3.44 (t, J=5.7 Hz, 2H, H8), 3.48-3.69 (m, 10H, H9-H13), 4.20 (t, J=6.3 Hz, 1H, H9″), 4.31-4.46 (m, 1H, H2), 4.38 (d, J=6.0 Hz, 2H, CH₂), 5.52 (d, J=7.8 Hz, 1H, NH), 7.29 (t, J=7.7 Hz, 2H, H2″ & H7″), 7.37 (t, J=7.4 Hz, 2H, H3″ & H6″), 7.50-7.64 (m, 2H, H1″ & H8″), 7.74 (d, J=7.2 Hz, 2H, H4″ & H5″), OH not observed. LRMS (ESI⁺, MeOH): m/z 536.2 (M+Na)⁺, C₂₉H₃₉NO₇Na⁺ requires 536.3.

### Example 5

### General Procedure for the Preparation of an Amino Acid Analogue Using N-Fmoc Butenylglycine 39 as Starting Material

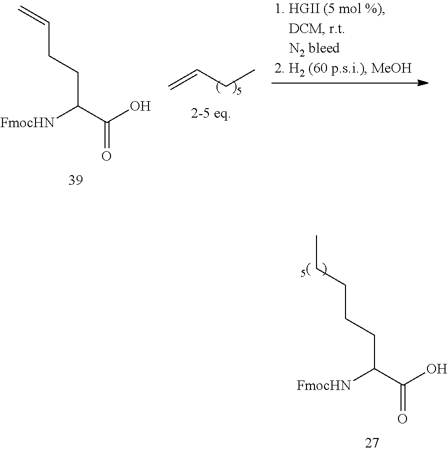

In a dry box under N₂ atmosphere, a Schlenk vessel equipped with a magnetic stir bar was charged with N-Fmoc allylglycine (100 mg, 0.296 mmol), degassed DCM (6.0 mL), terminal alkene (1.48 mmol, 5 eq.) and HGII (9.29 mg, 5 mol %). The vessel was sealed, removed from the dry box and attached to a vacuum manifold. The vessel was placed under a flow of nitrogen and the quick fit stopper replaced with a suba seal pierced with a 26 gauge needle to allow a constant flow of nitrogen over the top of the reaction. The reaction was stirred at room temperature overnight allowing all of the DCM to evaporate. The residue was washed with hexane (2×10 mL) and collected via filtration or centrifuge. The residue was then re-dissolved in MeOH (10 mL) and transferred to a Fischer-Porter tube. The vessel was charged with H₂ (60 p.s.i.), sealed and stirred at room temperature overnight. The reaction mixture was then concentrated in vacuo and the residual brown solid was purified via column chromatography (EtOAc:Hexane:AcOH=3:1:0.05) to give the title compound as a colourless solid (103.6 mg, 57%).

Compound Characterisation

While the amino acid analogues produced by the general procedure described above may be used (e.g. incorporated into a peptide sequence) without further purification, each of the following amino acid analogues were purified for by column chromatography for characterisation purposes.

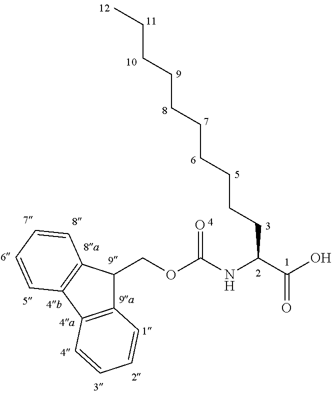 [SMILES: CCCCCCCCCC[C@H](NC(=O)OCC1c2ccccc2-c2ccccc21)C(=O)O]

m.p. 61-62° C. ¹H n.m.r. (400 MHz, CDCl₃): δ 0.88 (3H, t, J=6.8 Hz, H12), 1.26-1.37 (16H, m, H4-11), 1.64-1.93 (2H, m, H3), 4.22 (1H, t, J=6.8 Hz, H2), 4.37-4.48 (1H, m, CH), 4.41 (2H, d, J=6.8 Hz, CH₂), 5.31 (1H, d, J=8.0 Hz, NH), 7.30 (t, J=7.4 Hz, 2H, H2′ & H7′), 7.39 (t, J=7.4 Hz, 2H, H3′ & H6′), 7.57-7.60 (m, 2H, H1′ & H8′), 7.75 (d, J=7.4 Hz, 2H, H4′ & H5′), OH not observed. LRMS (ESI⁺, MeOH): m/z 460.2 (M+Na)⁺, C₁₇H₃₅NO₄Na requires 460.25. Spectral data are in agreement with compound 27.

In this specification, including the claims which follow, except where the context requires otherwise due to express language or necessary implication, the word “comprising” or variations such as “comprise” or “comprises” is used in the inclusive sense, to specify the presence of the stated features or steps but not to preclude the presence or addition of further features or steps.

It is to be understood that, if any prior art publication is referred to herein, such reference does not constitute an admission that the publication forms a part of the common general knowledge in the art, in Australia or any other country.

1. A method for the synthesis of an amino acid analogue or a salt, solvate, derivative, isomer or tautomer thereof comprising the steps of:
(i) subjecting an amino acid containing a metathesisable group to metathesis with a compound containing a complementary metathesisable group of formula (I) or (II): 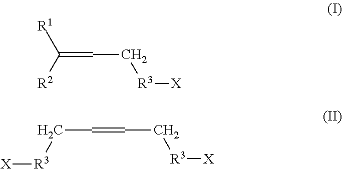
wherein R¹ and R² are independently selected from H and substituted or unsubstituted C₁ to C₄ alkyl; each R³ is either absent or independently selected from a heteroatom, a substituted or unsubstituted C₁ to C₂₀ alkyl, and a substituted or unsubstituted C₁ to C₂₀ alkyl group interrupted by one or more heteroatoms; and
each X is independently selected from H and an effector molecule;
in the presence of a reagent to catalyse the metathesis to form a dicarba bridge between the amino acid containing a metathesisable group and the compound containing a complementary metathesisable group; and
(ii) reducing the dicarba bridge to form a saturated dicarba bridge, wherein the reagent used to catalyse step (i) also catalyses step (ii).

2. The method of claim 1, wherein the metathesisable group of the amino acid is of the general formula (IV): 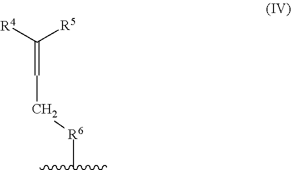
wherein R⁴ and R⁵ are independently selected from H and substituted or unsubstituted C₁ to C₄ alkyl; and R⁶ is either absent or selected from a heteroatom, a substituted or unsubstituted C₁ to C₂₀ alkyl, and a substituted or unsubstituted C₁ to C₂₀ alkyl interrupted by one or more heteroatoms.

3. The method of claim 1 or 2, wherein the amino acid having the metathesisable group is a compound of formula (VI): 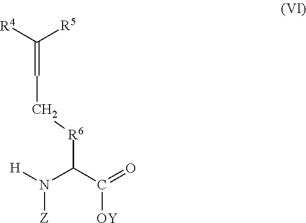
wherein R⁴ and R⁵ are independently selected from H and substituted or unsubstituted C₁ to C₄ alkyl; R⁶ is either absent or selected from a heteroatom, a substituted or unsubstituted C₁ to C₂₀ alkyl, and a substituted or unsubstituted C₁ to C₂₀ alkyl interrupted by one or more heteroatoms; Z is selected from H, a salt and a protecting group; and Y is selected from H and a protecting group.

4. The method of claim 3, wherein the compound of formula (VI) is selected from:
optionally protected allylglycine wherein Z is H or a protecting group, Y is H or a protecting group, R⁴ and R⁵ are H, and R⁶ is absent;
optionally protected crotylglycine wherein Z is H or a protecting group, Y is H or a protecting group, one of R⁴ and R⁵ is H and the other is methyl, and R⁶ is absent; or
optionally protected butenylglycine wherein Z is H or a protecting group, Y is H or a protecting group, R⁴ and R⁵ are H, and R⁶ is methylene.

5. The method of any one of claims 1 to 3, wherein the heteroatom is selected from group consisting of O, S(O), S(O)₂, SO₂NH, OS(O₂)O, NH, N(R⁷), PO₄, and P(R⁷)₂, wherein each R⁷ is independently a substituted or unsubstituted C₁ to C₁₀ alkyl.

6. The method of any one of claims 1 to 5, wherein each R³ is the same and each X is the same in the compound of formula (II).

7. The method of any one of claims 1 to 6, wherein the amino group of the amino acid containing a metathesisable group is protected during the metathesis reaction.

8. The method of any one of claims 1 to 7, wherein the reagent used to catalyse step (i) and step (ii) is a ruthenium alkylidene catalyst.

9. The method of claim 8, wherein the ruthenium alkylidene catalyst is a non-phosphine ruthenium alkylidene catalyst.

10. The method of claim 9, wherein the non-phosphine ruthenium alkylidene catalyst is a Hoveyda-Grubbs catalyst.

11. The method of any one of claims 1 to 10, wherein the reduction step (ii) is performed at a temperature below 100° C.

12. The method of any one of claims 1 to 11, wherein the reduction step (ii) is performed at a hydrogen pressure of 100 Psi or less.

13. The method of any one of claims 1 to 12, wherein the reduction step (ii) is performed in the absence of a base.

14. The method of any one of claims 1 to 13, wherein the reduction step (ii) is performed in the absence of any further catalyst.

15. An amino acid analogue or a salt, solvate, derivative, isomer or tautomer thereof synthesised by the method according to any one of claims 1 to 14.

16. A method for preparing a peptide containing an amino acid residue of formula (X¹) or a salt, solvate, derivative, isomer or tautomer thereof, 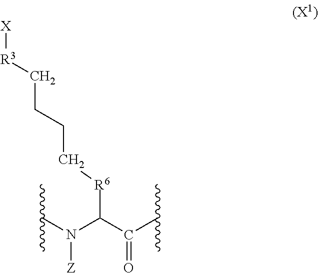
comprising the steps of:
(i) subjecting an amino acid containing a metathesisable group of formula (VI) to metathesis with a compound containing a complementary metathesisable group of formula (I) or (II): 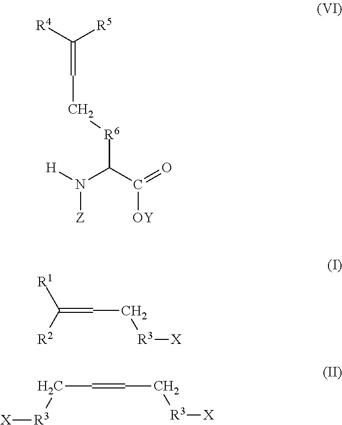
wherein R¹, R², R⁴ and R⁵ are independently selected from H and substituted or unsubstituted C₁ to C₄ alkyl; each R³ is either absent or independently selected from a heteroatom, a substituted or unsubstituted C₁ to C₂₀ alkyl, and a substituted or unsubstituted C₁ to C₂₀ alkyl group interrupted by one or more heteroatoms; R⁶ is either absent or selected from a heteroatom, a substituted or unsubstituted C₁ to C₂₀ alkyl, and a substituted or unsubstituted C₁ to C₂₀ alkyl interrupted by one or more heteroatoms; Z is selected from H, a salt and a protecting group; Y is selected from H and a protecting group; and each X is independently selected from H and an effector molecule;
in the presence of a reagent to catalyse the metathesis to form a dicarba bridge between the amino acid of formula (VI) and the compound of formula (I) or (II);
(ii) reducing the dicarba bridge to form a saturated dicarba bridge, wherein the reagent used to catalyse step (i) also catalyses step (ii); and
(iii) synthesising a peptide by stepwise addition of amino acid residues to produce the peptide, wherein one or more of the amino acid residues is of formula (X¹).

17. A peptide or a salt, solvate, derivative, isomer or tautomer thereof synthesised by the method according to claim 16.

18. A method for the synthesis of a peptide or peptides containing a dicarba bridge or a salt, solvate, derivative, isomer or tautomer thereof comprising the steps of:
(i) subjecting a reactable peptide containing a metathesisable group to metathesis with a compound containing a complementary metathesisable group of formula (I′) or (II′): 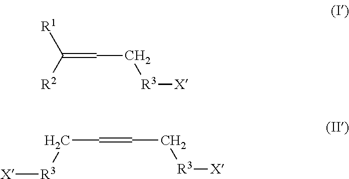
wherein R¹ and R² are independently selected from H and substituted or unsubstituted C₁ to C₄ alkyl; each R³ is either absent or independently selected from a heteroatom, a substituted or unsubstituted C₁ to C₂₀ alkyl, and a substituted or unsubstituted C₁ to C₂₀ alkyl group interrupted by one or more heteroatoms; and each X′ is independently selected from H, an effector molecule, an amino acid and a peptide;
in the presence of a reagent to catalyse the metathesis to form a dicarba bridge between the reactable peptide containing a metathesisable group and the compound containing a complementary metathesisable group; and
(ii) reducing the dicarba bridge to form a saturated dicarba bridge,
wherein the reagent used to catalyse step (i) also catalyses step (ii).

19. A method for the synthesis of a peptide containing a dicarba bridge or a salt, solvate, derivative, isomer or tautomer thereof comprising the steps of:
(i) subjecting a reactable peptide containing at least two metathesisable groups to metathesis in the presence of a reagent to catalyse the metathesis to form a dicarba bridge between the metathesisable groups of the reactable peptide; and
(ii) reducing the dicarba bridge to form a saturated dicarba bridge,
wherein the reagent used to catalyse step (i) also catalyses step (ii).

20. The method of claim 18 or 19, wherein the metathesisable group or groups of the reactable peptide is of the general formula (IV): 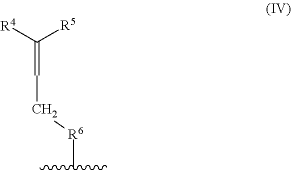
wherein R⁴ and R⁵ are independently selected from H and substituted or unsubstituted C₁ to C₄ alkyl; and R⁶ is either absent or selected from a heteroatom, a substituted or unsubstituted C₁ to C₂₀ alkyl, and a substituted or unsubstituted C₁ to C₂₀ alkyl interrupted by one or more heteroatoms.

21. The method of claim 18 or 19, wherein the reactable peptide containing a metathesisable group or groups is a peptide containing at least one compound of formula (VI¹): 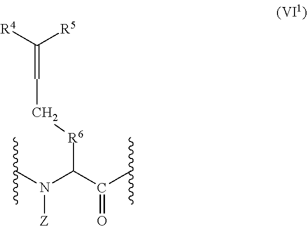
wherein R⁴ and R⁵ are independently selected from H and substituted or unsubstituted C₁ to C₄ alkyl; R⁶ is either absent or selected from a heteroatom, a substituted or unsubstituted C₁ to C₂₀ alkyl, and a substituted or unsubstituted C₁ to C₂₀ alkyl interrupted by one or more heteroatoms; and Z is selected from H, a salt and a protecting group.

22. The method of claim 21, wherein the compound of formula (VI¹) is selected from:
optionally protected allylglycine wherein Z is H or a protecting group, Y is H or a protecting group, R⁴ and R⁵ are H, and R⁶ is absent;
optionally protected crotylglycine wherein Z is H or a protecting group, Y is H or a protecting group, one of R⁴ and R⁵ is H and the other is methyl, and R⁶ is absent; or
optionally protected butenylglycine wherein Z is H or a protecting group, Y is H or a protecting group, R⁴ and R⁵ are H, and R⁶ is methylene.

23. The method of claim 18, wherein the compound containing a complementary metathesisable group of formula (I) or (II) is a compound of formula (XI) or a peptide containing a compound of formula (XII): 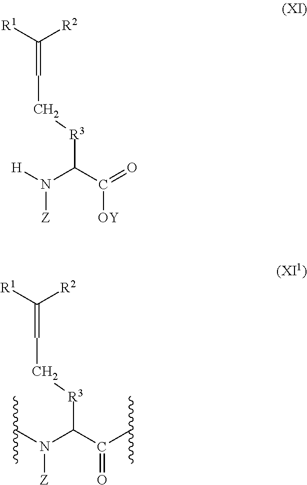
wherein R¹ and R² are independently selected from H and substituted or unsubstituted C₁ to C₄ alkyl; R³ is either absent or selected from a heteroatom, a substituted or unsubstituted C₁ to C₂₀ alkyl, and a substituted or unsubstituted C₁ to C₂₀ alkyl interrupted by one or more heteroatoms; Z is selected from H, a salt and a protecting group; and Y is selected from H and a protecting group.

24. The method of any one of claims 18 to 23, wherein the heteroatom is selected from group consisting of O, S(O), S(O)₂, SO₂NH, OS(O₂)O, NH, N(R⁷), PO₄, and P(R⁷)₂, wherein each R⁷ is independently a substituted or unsubstituted C₁ to C₁₀ alkyl.

25. The method of claim 18, wherein each R³ is the same and each X′ is the same in the compound of formula (II′).

26. The method of any one of claims 18 to 25, wherein the amino group of the amino acid containing a metathesisable group is protected during the metathesis reaction.

27. The method of any one of claims 18 to 26, wherein the reagent used to catalyse step (i) and step (ii) is a ruthenium alkylidene catalyst.

28. The method of claim 27, wherein the ruthenium alkylidene catalyst is a non-phosphine ruthenium alkylidene catalyst.

29. The method of claim 28, wherein the non-phosphine ruthenium alkylidene catalyst is a Hoveyda-Grubbs catalyst.

30. The method of any one of claims 18 to 29, wherein the reduction step (ii) is performed at a temperature below 100° C.

31. The method of any one of claims 18 to 30, wherein the reduction step (ii) is performed at a hydrogen pressure of 100 Psi or less.

32. The method of any one of claims 18 to 31, wherein the reduction step (ii) is performed in the absence of a base.

33. The method of any one of claims 18 to 32, wherein the reduction step (ii) is performed in the absence of any further catalyst.

34. A peptide or peptides containing a dicarba bridge or a salt, solvate, derivative, isomer or tautomer thereof, synthesised by the method according to any one of claims 18 to 33.
